(19) United States
(12) Patent Application Publication
(10) Pub. No.: US 2026/0024644 A1
(43) Pub. Date: Jan. 22, 2026

(54) PREDICTION OF METRICS OF MEDICAL PROCEDURES AND MEDICAL ENVIRONMENTS USING ROBOTIC SYSTEM DATA

(71) Applicant: Intuitive Surgical Operations, Inc., Sunnyvale, CA (US)

(72) Inventors: Reza Khodayi Mehr, Sunnyvale, CA (US); Muhammad Abdullah Jamal, Sunnyvale, CA (US); Brian E. Miller, Monte Sereno, CA (US); Omid Mohareri, San Francisco, CA (US); William C. Nowlin, Los Altos Hills, CA (US)

(73) Assignee: Intuitive Surgical Operations, Inc., Sunnyvale, CA (US)

(21) Appl. No.: 19/257,020

(22) Filed: Jul. 1, 2025

Related U.S. Application Data
(60) Provisional application No. 63/673,097, filed on Jul. 18, 2024.

(51) Int. Cl. G16H 20/40; A61B 34/00; A61B 34/30

(52) U.S. Cl. CPC G16H 20/40; A61B 34/25; A61B 34/30

(57) ABSTRACT

The arrangements disclosed herein relate to determining, via at least one machine learning model, a metric value using first robotic system data. The first robotic system data is generated by a first robotic system used to perform a first medical procedure in a first medical environment. The at least one machine learning model is based at least in part on second robotic system data and efficiency-related data comprising one or more second metric values for second medical procedures in second medical environments. The second robotic system data is generated by second robotic systems used to perform the second medical procedures in the second medical environments. The one or more second metric values is determined based at least in part on the second robotic system data and medical environment data generated by sensors deployed within the plurality of second medical environments. The metric value is provided for display on a User Interface (UI).

### CROSS-REFERENCE TO RELATED APPLICATION

This application claims priority to U.S. Patent Application No. 63/673,097, filed Jul. 18, 2024, the full disclosure of which is incorporated herein by reference in its entirety.

### TECHNICAL FIELD

Various of the disclosed embodiments relate to systems, apparatuses, methods, and non-transitory computer-readable media for predicting metrics of medical procedures and medical environments using robotic system data.

### BACKGROUND

Modern medical robotic systems can perform medical procedures (e.g., surgeries) in medical environments (e.g., operating rooms (ORs)) as controlled by a surgeon or even autonomously.

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation, as may occur in relation to some embodiments;

FIG. 1B is a schematic view of various elements appearing in a surgical theater during a surgical operation employing a robotic surgical system, as may occur in relation to some embodiments;

FIG. 2A is a schematic depth map rendering from an example theater-wide sensor perspective, as may be used in some embodiments;

FIG. 2B is a schematic top-down view of objects in the theater of FIG. 2A, with corresponding sensor locations;

FIG. 2C is a pair of images depicting a grid-like pattern of orthogonal rows and columns in perspective, as captured from a theater-wide visual image sensor having a rectilinear view and a theater-wide visual image sensor having a fisheye view, each of which may be used in connection with some embodiments;

FIG. 3 is a schematic representation of a series of surgical procedures within a surgical theater, their intervening nonoperative periods, and corresponding theater-wide sensor datasets for one such nonoperative period, as may occur in connection with some embodiments;

FIG. 4 is a schematic block diagram illustrating an example deployment topology for a nonoperative periods analysis system, as may be implemented in some embodiments;

FIG. 5A is a schematic representation of a collection of metrics intervals, as may be used to assess nonoperative team performance in some embodiments;

FIG. 5B is a schematic processing diagram indicating full-day relations of various intervals, including intervals from FIG. 5A, as may be applied in some embodiments;

FIG. 5C is a schematic block diagram indicating possible activity analysis class groupings, as may be used in connection with some embodiments;

FIG. 6 is a table of example task action temporal definitions, as may be used in some embodiments;

FIG. 7 is a table of additional example task action temporal definitions, as may be used in some embodiments;

FIG. 8 is a schematic block diagram illustrating various metrics and their relation in constructing a composite score (referred to as an OR analysis “ORA” score), as may be used in some embodiments;

FIG. 9A is a schematic block diagram depicting a general nonoperative analysis system processing flow, as may be implemented in some embodiments;

FIG. 9B is a schematic block diagram depicting elements in a more detailed example nonoperative analysis system processing flow than the flow depicted in FIG. 9A, as may be implemented in some embodiments;

FIG. 9C is a flow diagram illustrating various operations in an example overall process for analyzing theater-wide sensor data during nonoperative periods, as may be implemented in some embodiments;

FIG. 10 is a flow diagram illustrating various operations in an example nonoperative segment detection process, as may be performed in some embodiments;

FIG. 11A is a schematic block diagram illustrating an example information processing flow for performing object detection, as may be used in connection with some embodiments;

FIG. 11B is a flow diagram illustrating various operations in an example process for performing object detection, as may be used in some embodiments;

FIG. 12A is schematic block diagram illustrating an example object tracking information processing flow, as may be used in connection with some embodiments;

FIG. 12B is flow diagram illustrating various operations in an example process for performing object tracking, as may be used in connection with some embodiments;

FIG. 13A is a schematic visual image and depth frame theater-wide data pair, from theater-wide data video, with an indication of the optical-flow derived correspondence, as may be used in some embodiments;

FIG. 13B is a schematic top-down view of the scene depicted in FIG. 13A;

FIG. 13C is a schematic pair of visual images showing team member motion distant from and near to an imaging sensor;

FIG. 13D three-dimensional is a schematic top-down view depicting the team member motion presented in the visual images of FIG. 13C;

FIG. 14 is a flow diagram illustrating various operations in an example process for performing motion analysis of nonoperative periods from theater-wide data, as may be used in connection with some embodiments;

FIG. 15 is flow diagram illustrating various operations in an example process for performing clustering and outlier determination analysis based upon metric values, such as those disclosed herein, as may be performed in some embodiments;

FIG. 16 is flow diagram illustrating various operations in an example process for providing coaching feedback based upon determined metric values, as may be performed in some embodiments;

FIG. 17 is a schematic representation of graphical user interface (GUI) elements in an example dashboard interface layout for nonoperative metrics quick review, as may be implemented in some embodiments;

FIG. 18A is a schematic representation of a GUI element in an example global nonoperative metrics quick review dashboard, as may be implemented in some embodiments;

FIG. 18B is a schematic representation of arrow graphical elements, as may be used in, e.g., the element of FIG. 18A in some embodiments;

FIG. 18C is a schematic representation of an example global nonoperative metrics quick review dashboard layout, as may be implemented in some embodiments;

FIG. 19A is a plot of example interval metric values acquired in connection with an example prototype implementation of an embodiment;

FIG. 19B is a plot of example interval metric values as acquired in connection with an example prototype implementation of an embodiment;

FIG. 20A is a plot of example interval metric values as acquired in connection with an example prototype implementation of an embodiment;

FIG. 20B is a plot of example interval metric values as acquired in connection with an example prototype implementation of an embodiment;

FIG. 21A is a plot of example metric values as acquired in connection with an example prototype implementation of an embodiment;

FIG. 21B is a plot of example metric values as acquired in connection with an example prototype implementation of an embodiment;

FIG. 21C is a plot of example metric values as acquired in connection with an example prototype implementation of an embodiment;

FIG. 22 is a schematic representation of example GUI elements for providing metrics-derived feedback, as may be used in some embodiments;

FIG. 23 is a block diagram of an example prediction system, according to some embodiments;

FIG. 24 is an example timeline including robotic system data over time, according to some embodiments;

FIG. 25 is an example timeline including various segments, according to some embodiments;

FIG. 26A is a block diagram of an example method for determining correlations between efficiency-related data and robotic system data and using the correlations to determine predicted metrics, according to some embodiments;

FIG. 26B is a flowchart diagram of an example method for determining correlations between efficiency-related data and robotic system data and using the correlations to determine predicted metrics, according to some embodiments;

FIG. 27A is a block diagram of an example method for determining predicted metrics, according to some embodiments;

FIG. 27B is a flowchart diagram of an example method for determining predicted metrics, according to some embodiments;

FIG. 28A is a block diagram of an example method for determining predicted metrics, according to some embodiments;

FIG. 28B is a flowchart diagram of an example method for determining predicted metrics, according to some embodiments;

FIG. 29 illustrates an example UI configured to display a predicted metric and other associated information, according to some embodiments; and

FIG. 30 is a block diagram of an example computer system as may be used in conjunction with some of the embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

Accordingly, there exists a need for systems and methods to overcome challenges and difficulties such as those described above. For example, there exists a need for systems and methods to process disparate forms of surgical theater data acquired during nonoperative periods so as to facilitate reviewer analysis and feedback generation based upon team member inefficiencies identified therein.

### Example Surgical Theaters Overview

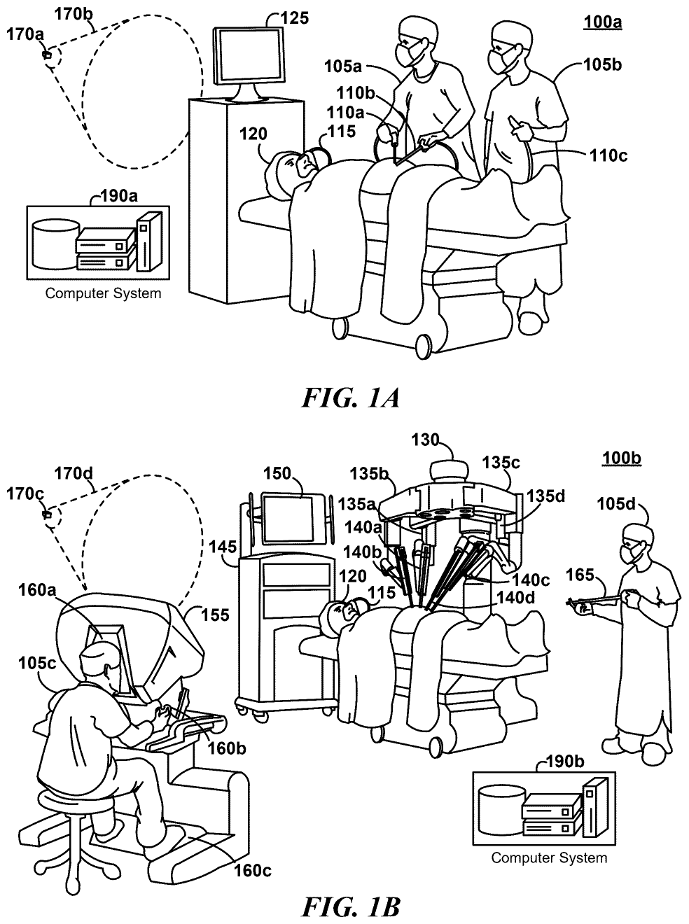

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound, visual image acquiring endoscope, etc., and a mechanical instrument 110a such as scissors, retractors, a dissector, etc.

The visualization tool 110b provides the surgeon 105a with an interior view of the patient 120, e.g., by displaying visualization output from an imaging device mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is a visual image acquiring endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears on-screen, etc. While two-dimensional video capture with visualization tool 110b may be discussed extensively herein, as when visualization tool 110b is a visual image endoscope, one will appreciate that, in some embodiments, visualization tool 110b may capture depth data instead of, or in addition to, two-dimensional image data (e.g., with a laser rangefinder, stereoscopy, etc.).

A single surgery may include the performance of several groups (e.g., phases or stages) of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a robotic surgical system, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, one or more of tools 140a, 140b, 140c, and 140d may include a visualization tool (here visualization tool 140d), such as a visual image endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, be introduced. As before, one or more assisting members 105d may now anticipate such changes, working with operator 105c to make any necessary adjustments as the surgery progresses.

Also similar to the non-robotic surgical theater 100a, the output from the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc., the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement with respect to display 160a, etc., may all be recorded. Similarly, patient side cart 130 may record tool activations (e.g., the application of radiative energy, closing of scissors, etc.), movement of instruments, etc., throughout the surgery. In some embodiments, the data may have been recorded using an in-theater recording device, which may capture and store sensor data locally or at a networked location (e.g., software, firmware, or hardware configured to record surgeon kinematics data, console kinematics data, instrument kinematics data, system events data, patient state data, etc., during the surgery).

Within each of theaters 100a, 100b, or in network communication with the theaters from an external location, may be computer systems 190a and 190b, respectively (in some embodiments, computer system 190b may be integrated with the robotic surgical system, rather than serving as a standalone workstation). As will be discussed in greater detail herein, the computer systems 190a and 190b may facilitate, e.g., data collection, data processing, etc.

Similarly, many of theaters 100a, 100b may include sensors placed around the theater, such as sensors 170a and 170c, respectively, configured to record activity within the surgical theater from the perspectives of their respective fields of view 170b and 170d. Sensors 170a and 170c may be, e.g., visual image sensors (e.g., color or grayscale image sensors), depth-acquiring sensors (e.g., via stereoscopically acquired visual image pairs, via time-of-flight with a laser rangefinder, structural light, etc.), or a multimodal sensor including a combination of a visual image sensor and a depth-acquiring sensor (e.g., a red-green-blue depth RGB-D sensor). In some embodiments, sensors 170a and 170c may also include audio acquisition sensors or sensors specifically dedicated to audio acquisition may be placed around the theater. The sensors 170a and 170c can be referred to as exocentric sensors. In some examples, additional egocentric sensors can be worn by medical personnel such as a head-worn depth-acquiring sensor or a visual sensor (e.g., an RGB visual camera) to address occlusion issues. Data from both exocentric and egocentric sensors can be referred to as theater-wide data. A plurality of such sensors may be placed within theaters 100a, 100b, possibly with overlapping fields of view and sensing range, to achieve a more holistic assessment of the surgery. For example, depth-acquiring sensors may be strategically placed around the theater so that their resulting depth frames at each moment may be consolidated into a single three-dimensional virtual element model depicting objects in the surgical theater. Examples of a three-dimensional virtual element model include a three-dimensional point cloud (also referred to as three-dimensional point cloud data). Similarly, sensors may be strategically placed in the theater to focus upon regions of interest. For example, sensors may be attached to display 125, display 150, or patient side cart 130 with fields of view focusing upon the patient 120's surgical site, attached to the walls or ceiling, etc. Similarly, sensors may be placed upon console 155 to monitor the operator 105c. Sensors may likewise be placed upon movable platforms specifically designed to facilitate orienting of the sensors in various poses within the theater.

As used herein, a “pose” refers to a position or location and an orientation of a body. For example, a pose refers to the translational position and rotational orientation of a body. For example, in a three-dimensional space, one may represent a pose with six total degrees of freedom. One will readily appreciate that poses may be represented using a variety of data structures, e.g., with matrices, with quaternions, with vectors, with combinations thereof, etc. Thus, in some situations, when there is no rotation, a pose may include only a translational component. Conversely, when there is no translation, a pose may include only a rotational component.

Similarly, for clarity, “theater-wide” sensor data refers herein to data acquired from one or more sensors configured to monitor a specific region of the theater (the region encompassing all, or a portion, of the theater) exterior to the patient, to personnel, to equipment, or to any other objects in the theater, such that the sensor can perceive the presence within, or passage through, at least a portion of the region of the patient, personnel, equipment, or other objects, throughout the surgery. Sensors so configured to collect such “theater-wide” data are referred to herein as “theater-wide sensors.” For clarity, one will appreciate that the specific region need not be rigidly fixed throughout the procedure, as, e.g., some sensors may cyclically pan their field of view so as to augment the size of the specific region, even though this may result in temporal lacunae for portions of the region in the sensor's data (lacunae which may be remedied by the coordinated panning or fields of view of other nearby sensors). Similarly, in some cases, personnel or robotics systems may be able to relocate theater-wide sensors, changing the specific region, throughout the procedure, e.g., to better capture different tasks. Accordingly, sensors 170a and 170c are theater-wide sensors configured to produce theater-wide data. “Visualization data” refers herein to visual image or depth image data captured from a sensor. Thus, visualization data may or may not be theater-wide data. For example, visualization data captured at sensors 170a and 170c is theater-wide data, whereas visualization data captured via visualization tool 140d would not be theater-wide data (for at least the reason that the data is not exterior to the patient).

### Example Theater-Wide Sensor Topologies

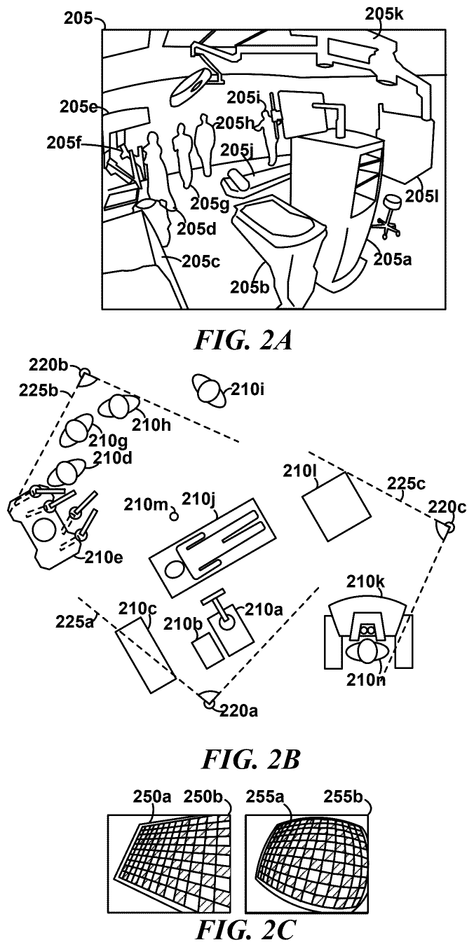

For further clarity regarding theater-wide sensor deployment, FIG. 2A is a schematic depth map rendering from an example theater-wide sensor perspective 205 as may be used in some embodiments. Specifically, this example depicts depth values corresponding to an electronics/control console 205a (e.g., the electronics/control console 145) and a nearby tray 205b, and cabinet 205c. Also within the field of view are depth values associated with a first technician 205d, presently adjusting a robotic arm (associated with depth values 205f) upon a robotic surgical system (associated with depth values 205e). Team members, with corresponding depth values 205g, 205h, and 205i, likewise appear in the field of view, as does a portion of the surgical table 205j. Depth values 205l corresponding to a movable dolly and a boom with a lighting system's depth values 205k also appear within the field of view.

The theater-wide sensor capturing the perspective 205 may be only one of several sensors placed throughout the theater. For example, FIG. 2B is a schematic top-down view of objects in the theater at a given moment during the surgical operation. Specifically, the perspective 205 may have been captured via a theater-wide sensor 220a with corresponding field of view 225a. Thus, for clarity, cabinet depth values 205c may correspond to cabinet 210c, electronics/control console depth values 205a may correspond to electronics/control console 210a, and tray depth values 205b may correspond to tray 210b. Robotic system 210e may correspond to depth values 205e, and each of the individual team members 210d, 210g, 210h, and 210i may correspond to depth values 205d, 205g, 205h, and 205i, respectively. Similarly, dolly 2101 may correspond to depth values 205l. Depth values 205j may correspond to table 210j (with an outline of a patient shown here for clarity, though the patient has not yet been placed upon the table corresponding to depth values 205j in the example perspective 205). A top-down representation of the boom corresponding to depth values 205k is not shown for clarity, though one will appreciate that the boom may likewise be considered in various embodiments.

As indicated, each of the sensors 220a, 220b, 220c is associated with different fields of view 225a, 225b, and 225c, respectively. The fields of view 225a-c may sometimes have complementary characters, providing different perspectives of the same object, or providing a view of an object from one perspective when it is outside, or occluded within, another perspective. Complementarity between the perspectives may be dynamic both spatially and temporally. Such dynamic character may result from movement of an object being tracked, but also from movement of intervening occluding objects (and, in some cases, movement of the sensors themselves). For example, at the moment depicted in FIGS. 2A and 2B, the field of view 225a has only a limited view of the table 210j, as the electronics/control console 210a substantially occludes that portion of the field of view 225a. Consequently, in the depicted moment, the field of view 225b is better able to view the surgical table 210j. However, neither field of view 225b nor 225a has an adequate view of the operator 210n in console 210k. To observe the operator 210n (e.g., when they remove their head in accordance with “head out” events), field of view 225c may be more suitable. However, over the course of the data capture, these complementary relationships may change. For example, before the procedure begins, electronics/control console 210a may be removed and the robotic system 210e moved into the position 210m. In this configuration, field of view 225a may instead be much better suited for viewing the patient table 210j than the field of view 225b. As another example, movement of the console 210k to the presently depicted pose of electronics/control console 210a may render field of view 225a more suitable for viewing operator 210n, than field of view 225c. Suitability of a field of view may thus depend upon the number and duration of occlusions, quality of the field of view (e.g., how close the object of interest is to the sensor), and movement of the object of interest within the theater. Such changes may be transitory and short in duration, as when a team member moving in the theater briefly occludes a sensor, or they may be chronic or sustained, as when equipment is moved into a fixed position throughout the duration of the procedure.

As mentioned, the theater-wide sensors may take a variety of forms and may, e.g., be configured to acquire visual image data, depth data, both visual and depth data, etc. One will appreciate that visual and depth image captures may likewise take on a variety of forms, e.g., to afford increased visibility of different portions of the theater. For example, FIG. 2C is a pair of images 250b, 255b depicting a grid-like pattern of orthogonal rows and columns in perspective, as captured from a theater-wide sensor having a rectilinear view and a theater-wide sensor having a fisheye view, respectively. More specifically, some theater-wide sensors may capture rectilinear visual images or rectilinear depth frames, e.g., via appropriate lenses, post-processing, combinations of lenses and post-processing, etc. while other theater-wide sensors may instead, e.g., acquire fisheye or distorted visual images or rectilinear depth frames, via appropriate lenses, post-processing, combinations of lenses and post-processing, etc. For clarity, image 250b depicts a checkboard pattern in perspective from a rectilinear theater wide sensor. Accordingly, the orthogonal rows and columns 250a shown here in perspective, retain linear relations with their vanishing points. In contrast, image 255b depicts the same checkboard pattern in the same perspective, but from a fish-eye theater-wise sensor perspective. Accordingly, the orthogonal rows and columns 255a, while in reality retaining a linear relationship with their vanishing points (as they appear in image 250b) appear here from the sensor data as having curved relations with their vanishing points. Thus, each type of sensor, and other sensor types, may be used alone, or in some instances, in combination, in connection with various embodiments.

Similarly, one will appreciate that not all sensors may acquire perfectly rectilinear, fisheye, or other desired mappings. Accordingly, checkered patterns, or other calibration fiducials (such as known shapes for depth systems), may facilitate determination of a given theater-wide sensor's intrinsic parameters. For example, the focal point of the fisheye lens, and other details of the theater-wide sensor (principal points, distortion coefficients, etc.), may vary between devices and even across the same device over time. Thus, it may be necessary to recalibrate various processing methods for the particular device at issue, anticipating the device variation when training and configuring a system for machine learning tasks. Additionally, one will appreciate that the rectilinear view may be achieved by undistorting the fisheye view once the intrinsic parameters of the camera are known (which may be useful, e.g., to normalize disparate sensor systems to a similar form recognized by a machine learning architecture). Thus, while a fisheye view may allow the system and users to more readily perceive a wider field of view than in the case of the rectilinear perspective, when a processing system is considering data from some sensors acquiring undistorted perspectives and other sensors acquiring distorted perspectives, the differing perspectives may be normalized to a common perspective form (e.g., mapping all the rectilinear data to a fisheye representation or vice versa).

### Example Surgical Theater Nonoperative Data

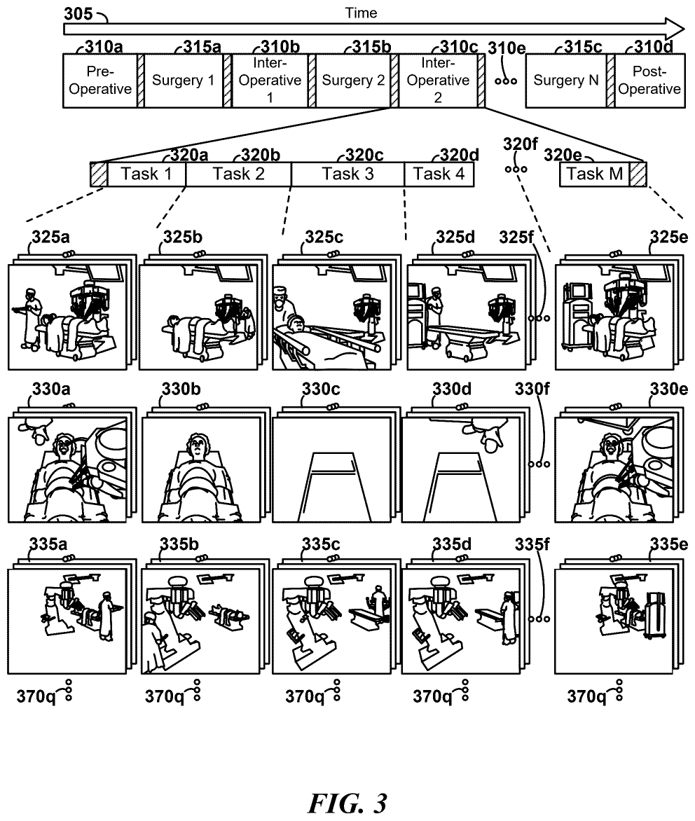

As discussed above, granular and meaningful assessment of team member actions and performance during nonoperative periods in a theater may reveal opportunities to improve efficiency and to avoid inefficient behavior having the potential to affect downstream operative and nonoperative periods. For context, FIG. 3 depicts a state of a single operating room over time 305, e.g., over the course of a day. In this example, during an initial pre-surgical period 310a, the team may prepare the operating room for the day's procedures, collecting appropriate equipment, reviewing scheduled tasks, etc. After performing the day's first surgery 315a, a nonoperative inter-operative period 310b will follow wherein the team performs the turnover from the operating room configuration for performing the surgery 315a to the configuration for performing the surgery 315b. Such alternating nonoperative and operative periods may continue throughout the day, e.g., nonoperative inter-surgical period 310c here follows the second surgery 315b, etc. After the final procedure 315c is performed for the day, the team may perform any final maintenance operations, may secure and put away equipment, deactivate devices, upload data, etc., during the post-operative period 310d. Ellipsis 310e indicates the possibility of additional intervening operative and nonoperative states (though, naturally, in some theaters there may instead by only one surgery during the day). Because of the theater operations' sequential character, an error in an upstream period can cause errors and delays to cascade through downstream periods. For example, improper alignment of equipment during pre-surgical period 310a may result in a delay during surgery 315a. This delay may itself require nonoperative period 310b to be shortened, providing a team member insufficient time to perform proper cleaning procedures, thereby placing the patient of surgery 315b's health at risk. Thus, inefficiencies early in the day may result in the delay, poor execution, or rescheduling of downstream actions. Conversely, efficiencies early in the day may provide tolerance downstream for unexpected events, facilitating more predictable operation outcomes and other benefits.

Each of the theater states, including both the operative periods 315a, 315b, etc. and nonoperative periods 310a, 310b, 310c, 310d, etc. may be divided into a collection of tasks. For example, the nonoperative period 310c may be divided into the tasks 320a, 320b, 320c, 320d, and 320c (with intervening tasks represented by ellipsis 320f). In this example, at least three theater-wide sensors were present in the OR, each sensor capturing at least visual image data (though one will appreciate that there may be fewer than three streams, or more, as indicated by ellipses 370q). Specifically, a first theater-wide sensor captured a collection of visual images 325a (e.g., visual image video) during the first nonoperative task 320a, a collection of visual images 325b during the second nonoperative task 320b, a collection of visual images 325c during the third nonoperative task 320c, a collection of visual images 325d during the fourth nonoperative task 320d, and the collection of visual images 325e during the last nonoperative task 320c (again, intervening groups of frames may have been acquired for other tasks as indicated by ellipsis 325f).

Contemporaneously during each of the tasks of the second nonoperative period 310c, the second theater-wide sensor may acquire the data collections 330a-e (ellipsis 330f depicting possible intervening collections), and the third theater-wide sensor may acquire the collections of 335a-c (ellipsis 335f depicting possible intervening collections). Thus, one will appreciate, e.g., that the data in sets 325a, 330a, and 335a may be acquired contemporaneously by the three theater-wide sensors during the task 320a (and, similarly, each of the other columns of collected data associated with each respective nonoperative task). Again, though visual images are shown in this example, one will appreciate that other data, such as depth frames, may alternatively, or additionally, be likewise acquired in each collection.

Thus, in task 320a, which may be an initial “cleaning” task following the surgery 315b, the sensor associated with collections 325a-e depicts a team member and the patient in a first perceptive. In contrast, the sensor capturing collections 335a-e is located on the opposite side of the theater and provides a fisheye view from a different perspective. Consequently, the second sensor's perception of the patient is more limited. The sensor associated with collections 330a-c is focused upon the patient, however, this sensor's perspective doesn't depict the team member very well in the collection 330a, whereas the collection 325a does provide a clear view of the team member.

Similarly, in task 320b, which may be a “roll-back” task, moving the robotic system away from the patient, the theater-wide sensor associated with collections 330a-e depicts that the patient is no longer subject to anesthesia, but does not depict the state of the team member relocating the robotic system. Rather, the collections 325b and 335b each depict the team member and the new pose of the robotic system at a point distant from the patient and operating table (though the sensor associated with the stream collections 335a-e is better positioned to observe the robot in its post-rollback pose).

In task 320c, which may be a “turnover” or “patient out” task, a team member escorts the patient out of the operating room. While the theater-wide sensor associated with collection 325c has a clear view of the departing patient, the theater-wide sensor associated with the collection 335c may be too far away to observe the departure in detail. Similarly, the collection 330c only indicates that the patient is no longer on the operating table.

In task 320d, which may be a “setup” task, a team member positions equipment which will be used in the next operative period (e.g., the final surgery 315c if there are no intervening periods in the ellipsis 310e).

Finally, in task 320e, which may be a “sterile prep” task before the initial port placements and beginning of the next surgery (again, e.g., surgery 315c), the theater-wide sensor associated with collection 330e is able to perceive the pose of the robotic system and its arms, as well as the state of the new patient. Conversely, collections 325e and 335e may provide wider contextual information regarding the state of the theater.

Thus, one can appreciate the holistic benefit of multiple sensor perspectives, as the combined views of the streams 325a-e, 330a-e, and 335a-e may provide overlapping situational awareness. Again, as mentioned, not all of the sensors may acquire data in exactly the same manner. For example the sensor associated with collections 335a-e may acquire data from a fisheye perspective, whereas the sensors associated with collections 325a-e and 330a-e may acquire rectilinear data. Similarly, there may be fewer or more theater-wide sensors and streams than are depicted here. Generally, because each collection is timestamped, it will be possible for a reviewing system to correlate respective streams' representations, even when they are of disparate forms. Thus, data directed to different theater regions may be reconciled and reviewed. Unfortunately, as mentioned, unlike periods 315a-c, surgical instruments, robotic systems, etc., may no longer be capturing data during the nonoperative periods (e.g., periods 310a-d). Accordingly, systems and reviewers regularly accustomed to analyzing the copious datasets available from periods 315a-c may find it especially difficult to review the more sparse data of periods 310a-d as they may need to rely only upon the disparate theater-wide streams 325a-e, 330a-e, and 335a-e. Even as the reader may have perceived in considering this figure, manually reconciling disparate, but contemporaneously captured perspectives, may be cognitively taxing upon a human reviewer.

### Example Nonoperative Activity Data Processing Overview

Various embodiments employ a processing pipeline facilitating analysis of nonoperative periods, and may include methods to facilitate iterative improvement of the surgical team's performance during these periods. Particularly, some embodiments include computer systems configured to automatically measure and analyze nonoperative activities in surgical operating rooms and recommend customized actionable feedback to operating room staff or hospital management based upon historical dataset patterns so as, e.g., to improve workflow efficiency. Such systems can also help hospital management assess the impact of new personnel, equipment, facilities, etc., as well as scale their review to a larger number, and more disparate types, of surgical theaters and surgeries, consequently driving down workflow variability. As discussed, various embodiments may be applied to surgical theaters having more than one modality, e.g., robotic, non-robotic laparoscopic, non-robotic open. Neither are various of the disclosed approaches limited to nonoperative periods associated with specific types of surgical procedures (e.g., prostatectomy, cholecystectomy, etc.).

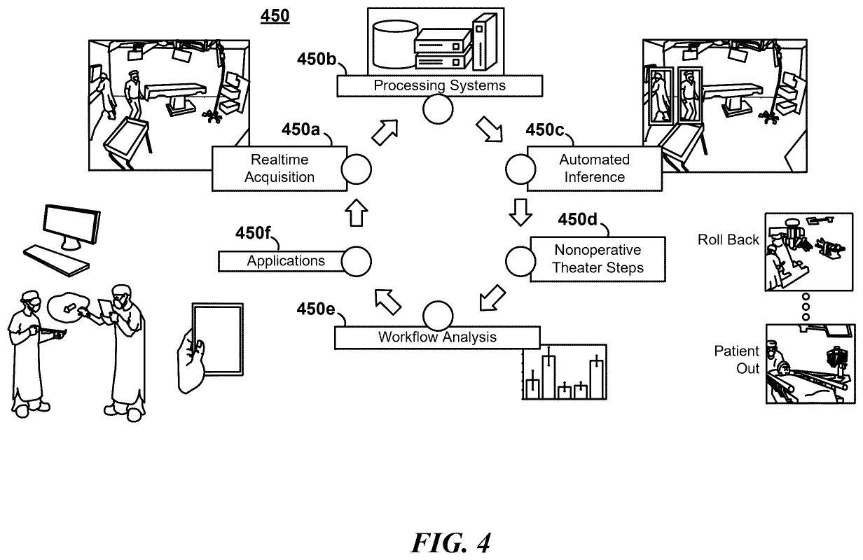

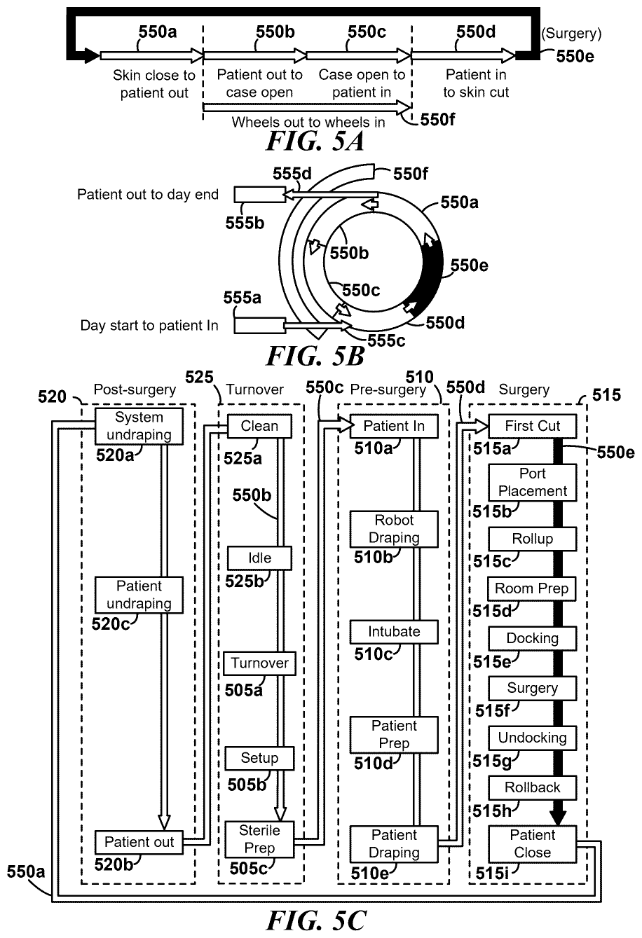

FIG. 4 is a schematic block diagram illustrating an example deployment topology 450 for a nonoperative periods analysis system of certain embodiments. As described herein, during realtime acquisition 450a, data may be collected from one or more theater-wide sensors in one or more perspectives. Multimodal (e.g., visual image and depth) sensor suites within a surgical theater (whether robotic or non-robotic) produce a wide variety of data. Consolidating this data into elemental and composite OR metrics, as described herein, may more readily facilitate analysis. To determine these metrics, the data may be provided to a processing systems 450b, described in greater detail herein, to perform automated inference 450c, including the detection of objects in the theater, such as personnel and equipment, as well as to segment the theater-wide data into distinct steps 450d (which may, e.g., correspond to the groupings and their respective actions discussed herein with respect to FIGS. 5A-C). The discretization of the theater-wide data into the steps 450d may facilitate more meaningful and granular determinations of metrics from the theater-wide data via various workflow analytics 450e, e.g., to ascertain surgical theater efficiency, to provide actionable coaching recommendations, etc.

Following the generation of such metrics during workflow analysis 450c, embodiments also disclose software and algorithms for presentation of the metric values along with other suitable information to users (e.g., consultants, students, medical staff, and so on) and for outlier detection within the metric values relative to historical patterns. As used herein, information of a plurality of medical procedures (e.g., procedure-related information or data, case-related information or data, information or data related to medical environments such as the ORs, and so on) refers to metric values and other associated information determined in the manners described herein. These analytics results may then be used to provide coaching and feedback via various applications 450f. Software applications 450f may present various metrics and derived analysis disclosed herein in various interfaces as part of the actionable feedback, a more rigorous and comprehensive solution than the prior use of human reviewers alone. One will appreciate that such applications 450f may be provided upon any suitable computer system, including desktop applications, tablets, augmented reality devices, etc. Such computer system can be located remote from the surgical theaters 100a and 100b in some examples. In other examples, such computer system can be located within the surgical theaters 100a and 100b (e.g., within the OR or the medical facility in which the hospital or OR processes occur). In one example, a consultant can review the information of a plurality of medical procedures via the applications 450f to provide feedback. In another example, a student can review the information of a plurality of medical procedures via the applications 450f to improve learning experience and to provide feedback. This feedback may result in the adjustment of the theater operation such that subsequent application of the steps 450a-f identify new or more subtle inefficiencies in the team's workflow. Thus, the cycle may continue again, such that the iterative, automated OR workflow analytics facilitate gradual improvement in the team's performance, allowing the team to adapt contextually based at least in part on upon the respective adjustments. Such iterative application may also help reviewers to better track the impact of the feedback to the team, analyze the effect of changes to the theater composition and scheduling, as well as for the system to consider historical patterns in future assessments and metrics generation.

### Example Nonoperative Interval Divisions

FIG. 5A is a schematic representation of a collection of metrics intervals as may be used to assess nonoperative team performance in some embodiments. One will appreciate that the intervals may be applied cyclically in accordance with the alternating character of the operative and nonoperative periods in the theater described above in FIG. 3. For example, initially, the surgical operation 315b may correspond to the interval 550e. Following the operation 315b's completion, actions and corresponding data in the theater may be allocated to consecutive intervals 550a-d during the subsequent nonoperative period 310c. Data and actions in the next surgery (e.g., surgery 315c, if there are no intervening periods in ellipsis 310e), may then be ascribed again to a second instance of the interval 550c, and so forth (consequently, data from each of the nonoperative periods 310b, 310b will be allocated to instances of intervals 550a-d). Intervals may also be grouped into larger intervals, as is the case here with the “wheels out to wheels in” interval 550f, which groups the intervals 550b and 550c, sharing the start time of interval 550b and the end time of interval 550c. Consolidating theater-wide data into this taxonomy, in conjunction with various other operations disclosed herein, may more readily facilitate analysis in a manner amenable to larger efficiency review, as described in greater detail herein. For example, organizing data in this manner may facilitate comparisons with different days of the week over the course of the month across theaters, surgery configurations (both robotic and non-robotic), and teams, with specific emphasis upon particular of these intervals 550a-d appearing in the corresponding nonoperative periods. Though not part of the nonoperative period, in some embodiments, it may still be useful to determine the duration of the surgery in interval 550e, as the duration may inform the efficiency or inefficiency of the preceding or succeeding nonoperative period. Accordingly, in some embodiments, some of the disclosed metrics may consider events and actions in this interval 550e, even when seeking ultimately to assess the efficiency of a nonoperative period.

For further clarity in the reader's understanding, FIG. 5B is a schematic block diagram indicating full-day relations of the elements from FIG. 5A. Specifically, as discussed above, instances of the intervals of FIG. 5A may be created cyclically in accordance with the alternating operative and nonoperative periods of FIG. 3. In some embodiments, when considering full day data (e.g., data including the nonoperative pre-operative period 310a, nonoperative post-operative period 310d, and all intervening periods), the system may accordingly anticipate a preliminary interval “day start to patient in” 555a to account for actions within the pre-operative period 310a. This interval may, e.g., begin when the first personnel enters the theater for the day and may end when the patient enters the theater for the first surgery. Accordingly, as shown by the arrow 555c, this may result in a transition to the first instance of the “patient in to skin cut” interval 550d. From there, as indicated by the circular relation, the data may be cyclically grouped into instances of the intervals 550a-c, e.g., in accordance with the alternating periods 315a, 310b, 315b, 310c, etc. until the period 315c.

At the conclusion of the final surgery for the day (e.g., surgery 315c), and following the last instance of the interval 550a after that surgery, then rather than continue with additional cyclical data allocations among instances of the intervals 550a-e, the system may instead transition to a final “patient out to day end” interval 555b, as shown by the arrow 555d (which may be used to assess nonoperative post-operative period 310d). The “patient out to day end” interval 555b may end when the last team member leaves the theater or the data acquisition concludes. One will appreciate that various of the disclosed computer systems may be trained to distinguish actions in the interval 555b from the corresponding data of interval 550b (naturally, conclusion of the data stream may also be used in some embodiments to infer the presence of interval 555b). Though concluding the day's actions, analysis of interval 555b may still be appropriate in some embodiments, as actions taken at the end of one day may affect the following day's performance.

### Example Task to Interval Assignments and Action Temporal Intervals

In some embodiments, the durations of each of intervals 550a-e may be determined based upon respective start and end times of various tasks or actions within the theater. Naturally, when the intervals 550a-e are used consecutively, the end time for a preceding interval (e.g., the end of interval 550c) may be the start time of the succeeding interval (e.g., the beginning of interval 550d). When coupled with a task action grouping ontology, theater-wide data may be readily grouped into meaningful divisions for downstream analysis. This may facilitate, e.g., consistency in verifying that team members have been adhering to proposed feedback, as well as computer-based verification of the same, across disparate theaters, team configurations, etc. As will be explained, some task actions may occur over a period of time (e.g., cleaning), while others may occur at a specific moment (e.g., entrance of a team member).

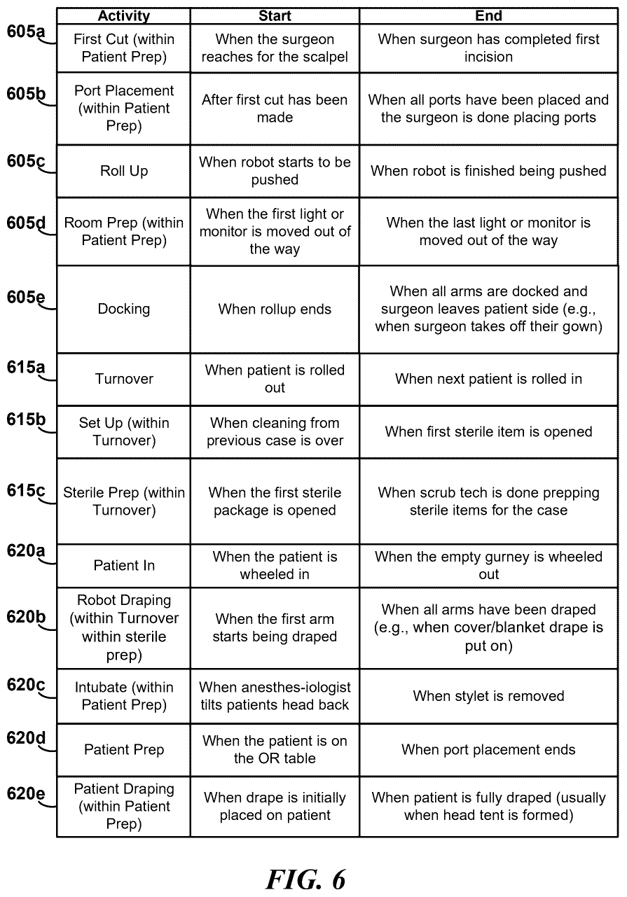

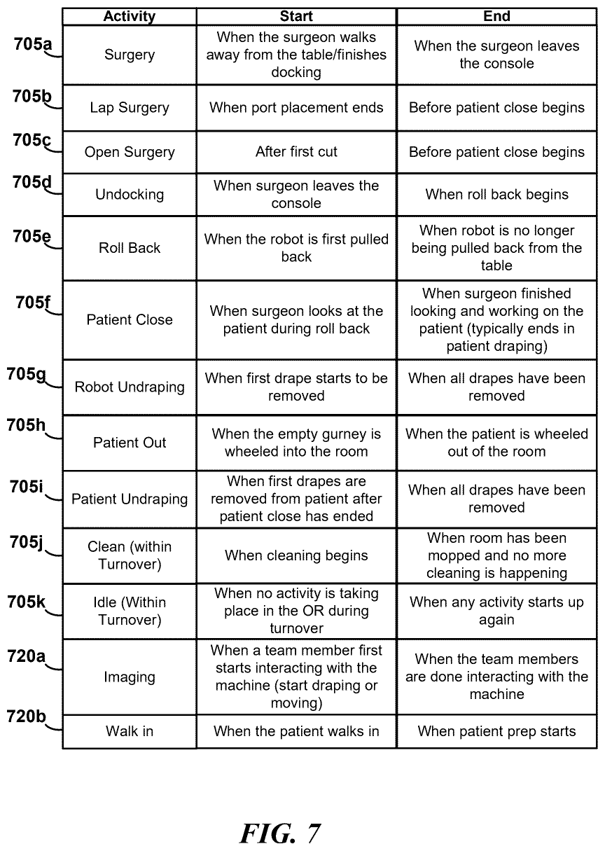

Specifically, FIG. 5C depicts four high-level task action classes or groupings of tasks, referred to for example as phases or stages: post-surgery 520, turnover 525, pre-surgery 510, and surgery 515. Surgery 515 may include the tasks or actions 515a-i. As will be discussed, FIGS. 6 and 7 provide various example temporal definitions for the actions, though for the reader's appreciation, brief summaries will be provided here. Specifically, the task “first cut” 515a, may correspond to a time when the first incision upon the patient occurs (consider, e.g., the duration 605a). The task “port placement” 515b, may correspond to a duration between the time when a first port is placed into the patient and the time when the last port is placed (consider, e.g., the duration 605b). The task “rollup” 515c, may correspond to the duration in which a team member begins moving a robotic system to a time when the robotic system assumes the pose it will use during at least an initial portion of the surgical procedure (consider, e.g., the duration 605c). The task “room prep” 515d, may correspond to a duration beginning with the first surgery preparation action specific to the surgery being performed and may conclude with the last preparation action specific to the surgery being performed (consider, e.g., the duration 605d). The task “docking” 515e, may correspond to a duration starting when a team member begins docking a robotic system and concludes when the robotic system is docked (consider, e.g., the duration 605e). The task “surgery” 515f, may correspond with a duration starting with the first incision and ending with the final closure of the patient (consider, e.g., the durations 705a-c for respective contemplated surgeries, specifically the robotic surgery 705a and non-robotic surgeries 705b and 705c). Naturally, in many taxonomies, these action blocks may be further broken down into considerably more action and task divisions in accordance with the analyst's desired focus (e.g., if the action “port placement” 515b were associated with an inefficiency, a supplemental taxonomy wherein each port's placement were a distinct action, with its own measured duration, may be appropriate for refining the analysis). Here, however, as nonoperative period actions are the subject of review, the general task “surgery” 515f (e.g., one of durations 705a-c) may suffice, despite surgery's encompassing many constituent actions. The task “undocking” 515g, may correspond to a duration beginning when a team member starts to undock a robotic system and concludes when the robotic system is undocked (consider, e.g., the duration 705d). The task “rollback” 515h, may correspond to a duration when a team member begins moving a robotic system away from a patient and concludes when the robotic system assumes a pose it will retain until turnover begins (consider, e.g., the duration 705c). The task “patient close” 515a, may correspond to a duration (e.g., duration 705f) when the surgeon observes the patient during rollback (e.g., one will appreciate by this example that some action durations may overlap and proceed in parallel).

Within the post-surgical class grouping 520, the task “robot undraping” 520a may correspond to a duration when a team member first begins undraping a robotic system and ends when the robotic system is undraped (consider, e.g., the duration 705g). The task “patient out” 520b, may correspond to a time, or duration, during which the patient leaves the theater (consider, e.g., the duration 705h). The task “patient undraping” 520c, may correspond to a duration beginning when a team member begins undraping the patient and ends when the patient is undraped (consider, e.g., the duration 705i).

Within the turnover class grouping 525, the task “clean” 525a, may correspond to a duration starting when the first team member begins cleaning equipment in the theater and concludes when the last team member (which may be the same team member) completes the last cleaning of any equipment (consider, e.g., the duration 705j). The task “idle” 525b, may correspond to a duration that starts when team members are not performing any other task and concludes when they begin performing another task (consider, e.g., the duration 705k). The task “turnover” 505a may correspond to a duration that starts when the first team member begins resetting the theater from the last procedure and concludes when the last team member (which may be the same team member) finishes the reset (consider, e.g., the duration 615a). The task “setup” 505b may correspond to a duration that starts when the first team member begins changing the pose of equipment to be used in a surgery, and concludes when the last team member (which may be the same team member) finishes the last equipment pose adjustment (consider, e.g., the duration 615a). The task “sterile prep” 505c, may correspond to a duration that starts when the first team member begins cleaning the surgical area and concludes when the last team member (which may be the same team member) finishes cleaning the surgical area (consider, e.g., the duration 615c). Again, while shown here in linear sequences, one will appreciate that task actions within the classes may proceed in orders other than that shown or, in some instances, may refer to temporal periods which may overlap and may proceed in parallel (e.g., when performed by different team members).

Within pre-surgery class grouping 510, the task “patient in” 510a may correspond to a duration that starts and ends when the patient first enters the theater (consider, e.g., the duration 620a). The task “robot draping” 510b may correspond to a duration that starts when the a member begins draping the robotic system and concludes when draping is complete (consider, e.g., the duration 620b). The task “intubate” 510c may correspond to a duration that starts when intubation of the patient begins and concludes when intubation is complete (consider, e.g., the duration 620c). The task “patient prep” 510d may correspond to a duration that starts when a team member begins preparing the patient for surgery and concludes when preparations are complete (consider, e.g., the duration 620d). The task “patient draping” 510e may correspond to a duration that starts when a team member begins draping the patient and concludes when the patient is draped (consider, e.g., the duration 620e).

Though not discussed herein, as mentioned, one will appreciate the possibility of additional or different task actions. For example, the durations of “Imaging” 720a and “Walk In” 720b, though not part of the example taxonomy of FIG. 5C, may also be determined in some embodiments.

Thus, as indicated by the respective arrows in FIG. 5C, the intervals of FIG. 5A may be allocated as follows. “Skin-close to patient-out” 550a may begin at the last closing operation 515j of the previous surgery interval and concludes with the patient's departure from the theater (e.g., from the end of the last suture at block 515i until the patient has departed at block 520b). Similarly, the interval “Patient-out to case-open” 550b may begin when the patient's departure from the theater at block 520b and concludes with the start of sterile prep at block 505c for the next case.

The interval “case-open to patient-in” 550c, may begin with the start of the sterile prep at block 505c and conclude with the start of the new patient entering the theater at block 510a. The interval “patient-in to skin cut” 550d may begin when the new patient enters the theater at block 510a and concludes at the start of the first cut at block 515. The surgery itself may occur during the interval 550e as shown.

As previously discussed, the “wheels out to wheels in” interval 550f is the interval from the start of “Patient out to case open” 550b and concludes with the end of “case open to patient in” 550c.

### Example Nonoperative Metric Generation and Scoring

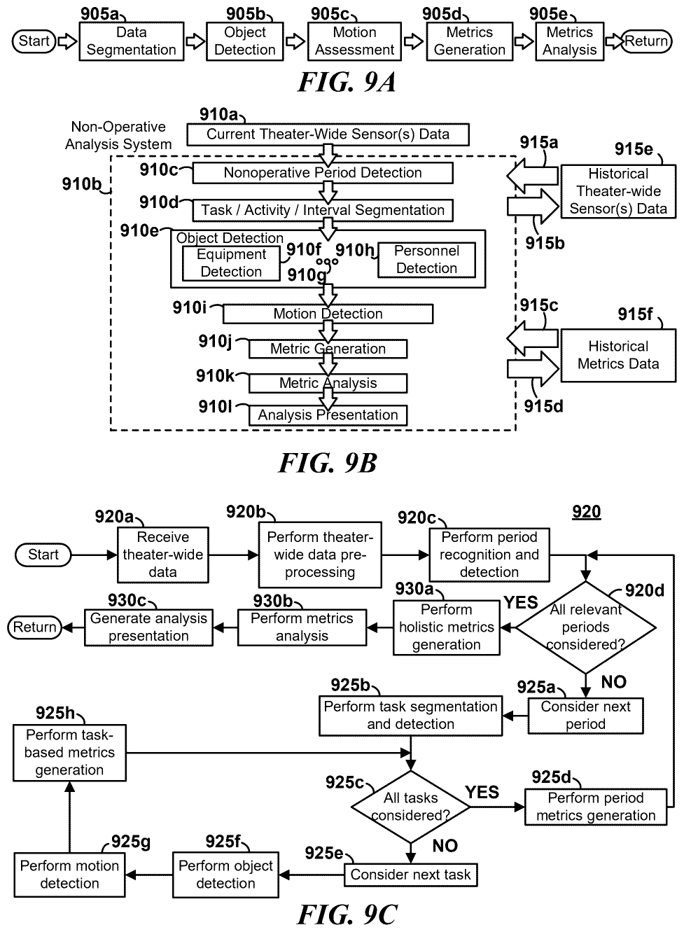

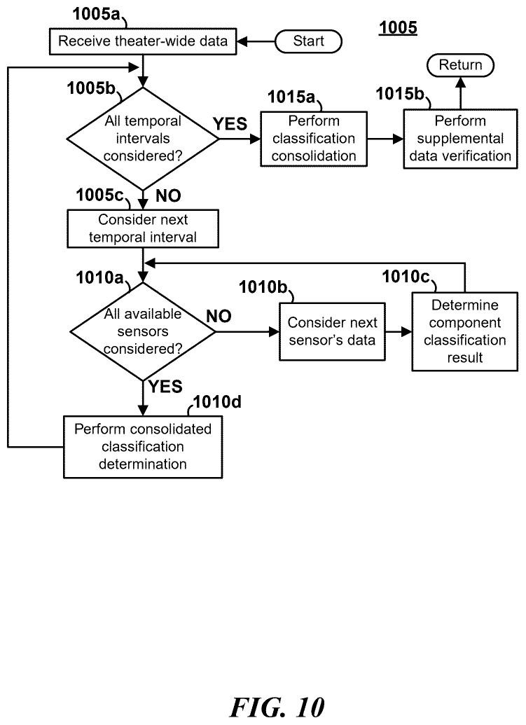

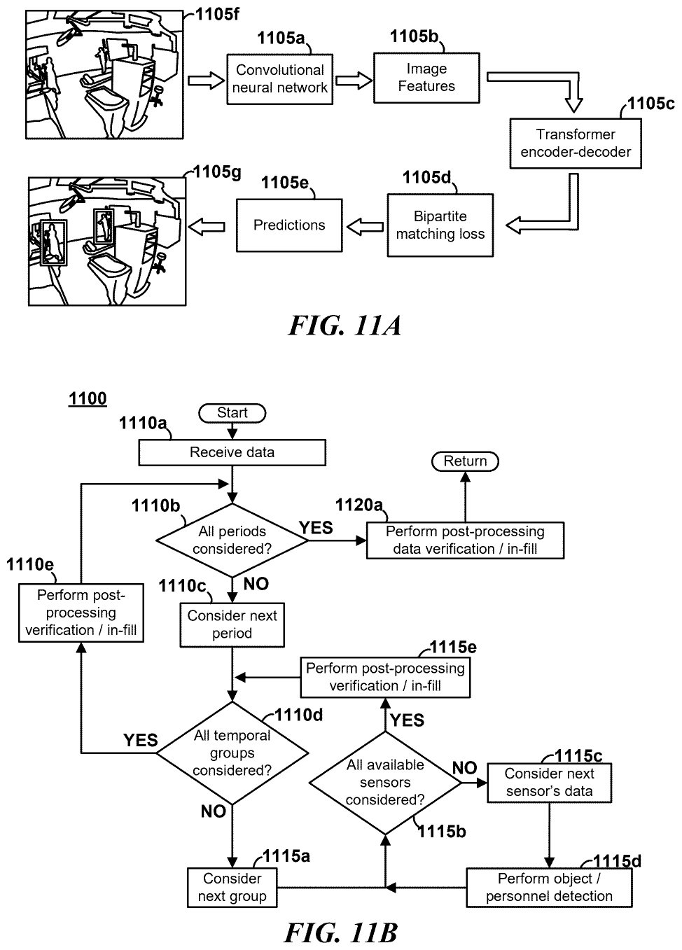

After the nonoperative segments have been identified (e.g., using systems and methods discussed herein with respect to FIGS. 9A-C and FIG. 10), the number and location of objects (e.g., using systems and methods discussed herein with respect to FIGS. 9A-C and FIGS. 11A-B), such as personnel, within each segment, and their respective motions have been identified (e.g., using systems and methods discussed herein with respect to FIGS. 9A-C, 12A-B, 13A-D, and 14), the system may generate one or more metric values. As mentioned, the duration and relative times of the intervals, classes, and task actions of FIGS. 5A-C may themselves serve as metrics.

Various embodiments may also determine “composite” metric scores based upon various of the other determined metrics. These metrics assume the functional form of EQN. 1:

$$s = f ( m ) \qquad ( 1 )$$

where s refers to the composite metric score value, which may be confined to a range, e.g., from 0 to 1, from 0 to 100, etc., and f(·) represents the mapping from individual metrics to the composite score. For example, m may be a vector of metrics computed using various data streams and models as disclosed herein. In such composite scores, in some embodiments, the constituent metrics may fall within one of temporal workflow, scheduling, human resource, or other groupings disclosed herein.

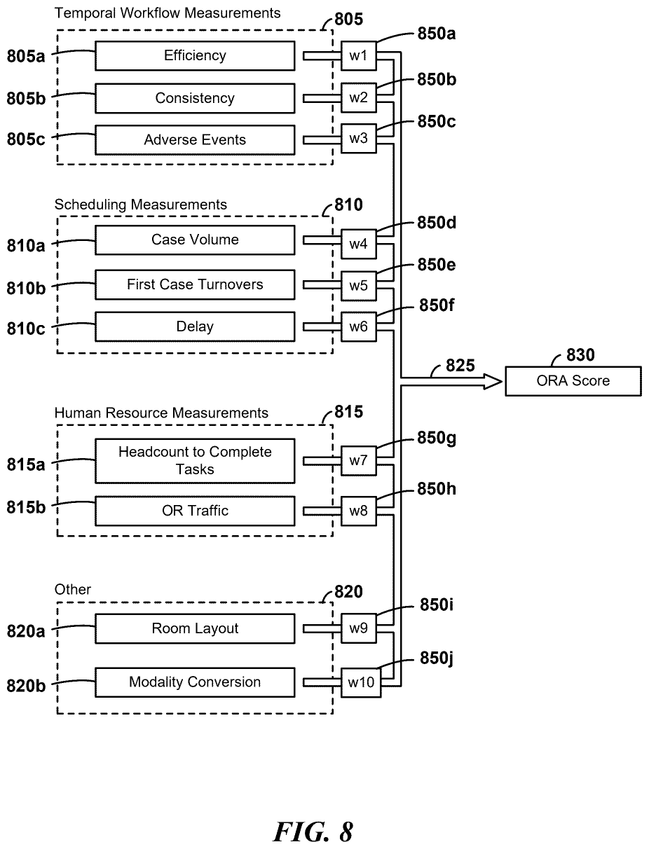

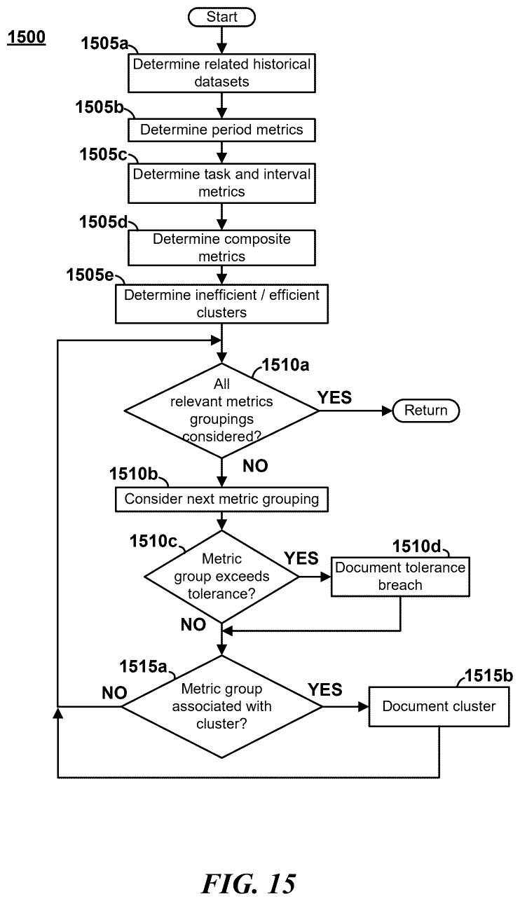

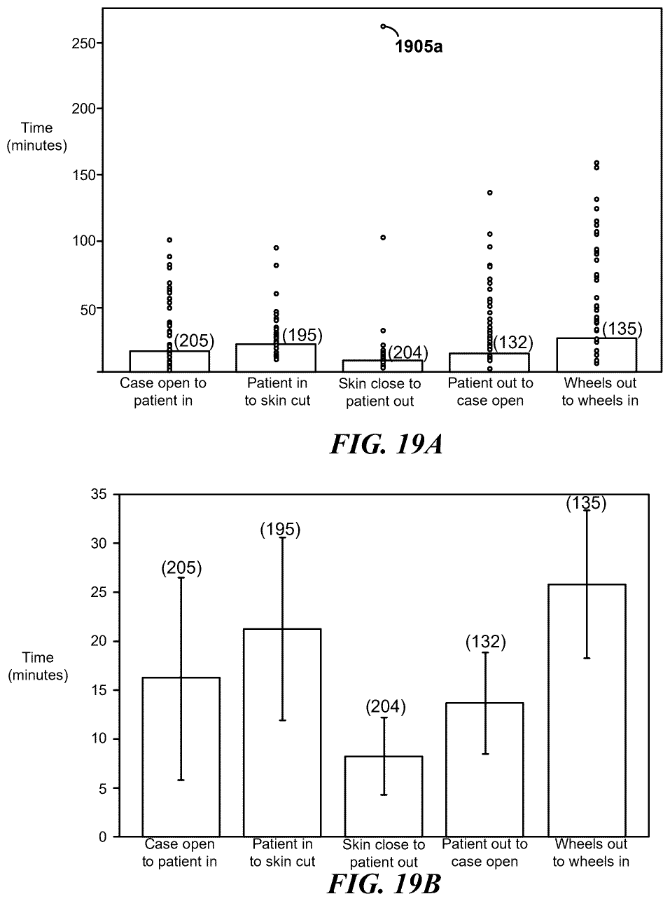

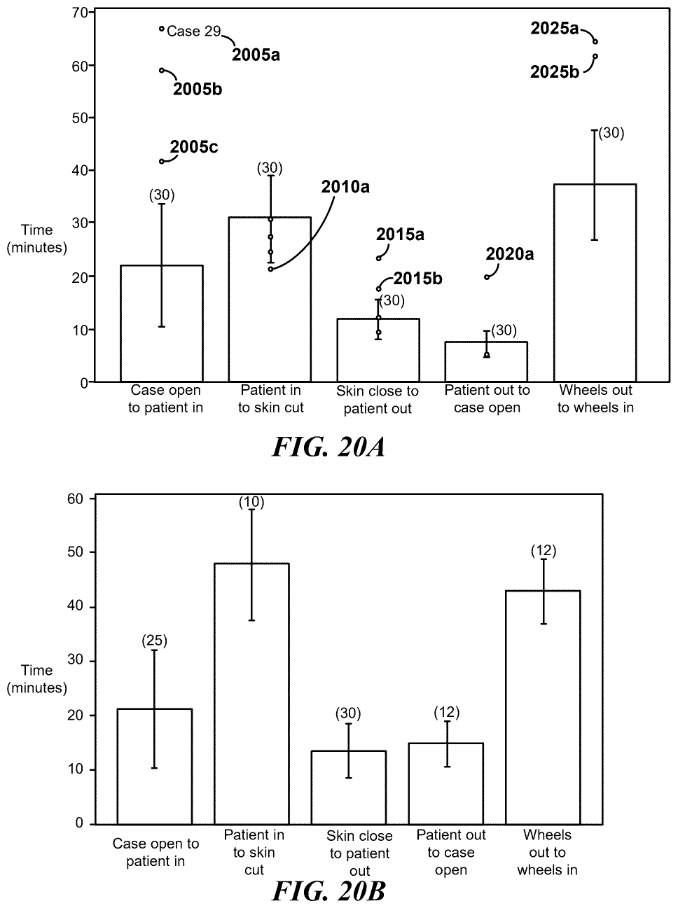

Specifically, FIG. 8 is a schematic block diagram illustrating various metrics and their relations in constructing an “ORA score” as may be performed in some embodiments. Within the temporal grouping 805, an “efficiency” scoring metric 805a may combine the nonoperative metrics that measure temporal workflow efficiency in an OR, e.g., the duration of one or more of the six temporal interval metrics of FIG. 5A. More specifically, the nonoperative metrics, averaged, as a mean or median, over all cases collected from a team, theater, or hospital, may be compared to the top 20% teams, theaters, or hospitals (e.g., as manually indicated by reviewers or from historical patterns via iterations of topology 450) in a database as a benchmark. A “consistency” metric 805b may combine (e.g., sum or find the mean or median) the standard deviations of nonoperative metrics (e.g., the six temporal interval metrics of FIG. 5A) across all cases collected from a current team, theater, or hospital. An “adverse event” metric 805c may combine (e.g., sum) negative outliers, e.g., as detected in terms of the interval metrics of FIGS. 5A-B. Outliers may, e.g., be detected using statistical analysis algorithms (e.g., clustering, distribution analysis, regression, etc. as discussed herein with reference to FIGS. 15, 16, 19A-B, and 20A-B). Negative outliers may be identified as those for which at least one of the nonoperative interval metrics of FIGS. 5A-B metrics are outside a threshold, such as a standard deviation, from than the relevant team, theater, or hospital median or mean (e.g., based at least in part on a threshold specified by an expert reviewer or upon historical patterns from past iterations of topology 450). Examples of such outliers are discussed herein, e.g., with respect to FIGS. 19A-B and FIGS. 20A-B.

Within the scheduling grouping 810, a “case volume” scoring metric 810a includes the mean or median number of cases operated per OR, per day, for a team, theater, or hospital, normalized by the expected case volume for a typical OR (e.g., again, as designated in a historical dataset benchmark, such as a mean or median). A “first case turnovers” scoring metric 810b is the ratio of first cases in an operating day that were turned over compared to the total number of first cases captured from a team, theater, or hospital. Alternatively, a more general “case turnovers” metric is the ratio of all cases that were turned-over compared to the total number of cases as performed by a team, in a theater, or in hospital. A “delay” scoring metric 810c is an mean or median positive (behind a scheduled start time of an action) or negative (before a scheduled start time of an action) departure from a scheduled time in minutes for each case, normalized by the acceptable delay (e.g., a historical mean or median benchmark). Naturally, the negative or positive definition may be reversed (e.g., wherein starting late is instead negative and starting early is instead positive) if other contextual parameters are likewise adjusted.

Within the human resource metrics grouping 815, a “headcount to complete tasks” scoring metric 815a combines the mean or median headcount (the largest number of detected personnel throughout the procedure in the OR at one time) over all cases collected for the team, theater, or hospital needed to complete each of the temporal nonoperative tasks for each case, normalized by the recommended headcount for each task (e.g., a historical benchmark median or mean). An “OR Traffic” scoring metric 815b measures the mean amount of motion in the OR during each case, averaged (itself as a median or mean) over all cases collected for the team, theater, or hospital, normalized by the recommended amount of traffic (e.g., based upon a historical benchmark as described above). For example, this metric may receive (two or three-dimensional) optical flow, and convert such raw data to a single numerical value, e.g., an entropy representation, a mean magnitude, a median magnitude, etc.

Within the “other” metrics grouping 815, a “room layout” scoring metric 820a includes a ratio of robotic cases with multi-part roll-ups or roll-backs, normalized by the total number of robotic cases for the team, theater, or hospital. That is, ideally, each roll up or back of the robotic system would include a single motion. When, instead, the team member moves the robotic system back and forth, such a “multi-part” roll implies an inefficiency, and so the number of such multi-part rolls relative to all the roll up and roll back events may provide an indication of the proportion of inefficient attempts. As indicated by this example, some metrics may be unique to robotic theaters, just as some metrics may be unique to nonrobotic theaters. Is some embodiments, correspondences between metrics unique to each theater-type may be specified to facilitate their comparison. A “modality conversion” scoring metric 820b includes a ratio of cases that have both robotic and non-robotic modalities normalized by the total number of cases for the team, theater, or hospital. For example, this metric may count the number of conversions, e.g., transitioning from a planned robotic configuration to a nonrobotic configuration, and vice versa, and then dividing the total number of such cases with such a conversion by the total cases. Whether occurring in an operative or nonoperative periods, such conversions may be reflective of inefficiencies in nonoperative periods (e.g., improper actions in a prior nonoperative period may have rendered the planned robotic procedure in the operative period impractical). Thus, this metric may capture inefficiencies in planning, in equipment, or in unexpected complications in the original surgical plan.

While each of the metrics 805a-c, 810a-c, 815a-c, and 820a-b may be considered individually to assess nonoperative period performances, or in combinations of the multiple of the metrics, as discussed above with respect to EQN. 1, some embodiments consider an “ORA score” 830 reflecting an integrated 825 representation of all these metrics. When, e.g., presented in combination with data of the duration of one or more of the intervals in FIG. 5A-C, the ORA score may provide a readily discernible means for reviewers to quickly and intuitively assess the relative performance of surgical teams, surgical theaters, hospitals and hospital systems, etc. during nonoperative periods, across theaters, across teams, across types of surgical procedures (nonoperative periods before or after prostatectomies, hernia repair, etc.), types of surgical modalities (nonoperative periods preparing for, or resetting after, nonrobotic laparoscopic procedures, nonrobotic open procedures, robotic procedures, etc.), hospital systems, etc.

Accordingly, while some embodiments may employ more complicated relationships (e.g., employing any suitable mathematical functions and operations) between the metrics 805a-c, 810a-c, 815a-c, and 820a-b in forming the ORA score 830, in this example, each of the metrics may be weighted by a corresponding weighting value 850a-j such that the integrating 825 is a weighted sum of each of the metrics. The weights may be selected, e.g., by a hospital administrator or reviewers in accordance with which of the metrics are discerned to be more vital to current needs for efficiency improvement. For example, in a system where reviewers wish to assess whether reports that limited staff are affecting efficiency, then the weight 850g may be upscaled relative to the other weights. Thus, when the ORA score 830 across procedures is compared in connection with the durations of one or more of the intervals in FIG. 5A-C for the groups of surgeries, the reviewer can more readily discern if there exists a relation between the head count and undesirable interval durations. Naturally, one will appreciate other choices and combinations of weight adjustment, as well as particular consideration of specific interval durations, to assess other performance characteristics.

### Example Metric Scoring Methodologies-ORA Significance Assessment

Some higher ORA composite metrics scores may positively correlate with increased system utilization u and reduced OR minutes per case t for the hospitals in a database, e.g., as represented by EQN. 2:

$${p}_{s , u} = \frac{cov ( s , u )}{{σ}_{s} {σ}_{u}} \geq 0.75 \qquad ( 2 )$$

Thus, the ORA composite score may be used for a variety of analysis and feedback applications. For example, the ORA composite score may be used to detect negative trends and prioritize hospitals, theaters, teams, or team members, that need workflow optimizations. The ORA composite score may also be used to monitor workflow optimizations, e.g., to verify adherence to requested adjustments, as well as to verify that the desired improvements are, in fact, occurring. The ORA composite score may also be used to provide an objective measure of efficiency for when teams perform new types of surgeries for the first time.

### Example Metric Scoring Methodologies-Additional Metrics

Additional metrics to assess workflow efficiency may be generated by compositing time, staff count, and motion metrics. For example, a composite score may consider scheduling efficiency (e.g., a composite formed from one or more of case volume 810a, first case turnovers 810b, and case delay 810c) and one or both of modality conversion 820b and the duration of an “idle time” metric, which is a mean or median of the idle time (for individual members or teams collectively) over a period (e.g., during action 525b).

Though, for convenience, sometimes described as considering the behavior of one or more team members, one will appreciate that the metrics described herein may be used to compare the performances of individual members, teams, theaters (across varying teams and modalities), hospitals, hospital systems, etc. Similarly, metrics calculated at the individual, team, or hospital level may be aggregated for assessments of a higher level. For example, to compare hospital systems, metrics for team members within each of the systems, across the system's hospitals, may be determined, and then averaged (e.g., a mean, median, sum weighted by characteristics of the team members, etc.) for a system-to-system comparison.

### Example Nonoperative Data Processing Workflow

FIG. 9A is a schematic block diagram depicting a general processing flow as may be implemented in some embodiments. Specifically, this example flow employs various machine learning consolidation systems for producing elemental OR metrics (such as temporal interval durations, personnel presence, personnel motion, equipment motion, etc., from which other metrics, e.g., as described in FIG. 8, may be generated) from the raw multimodal theater-wide sensor data.

In some embodiments (e.g., where the data has not been pre-processed), a nonoperative segment detection module 905a may be used to detect nonoperative segments from full-day theater-wide data. A personnel count detection module 905b may then be used to detect a number of people involved in each of the detected nonoperative segments/activities of the theater-wide data (e.g., a spatial-temporal machine learning algorithm employing a three-dimensional convolutional network for handing visual image and depth data over time, e.g., as appearing in video). A motion assessment module 905c may then be used to measure the amount of motion (e.g., people, equipment, etc.) observed in each of the nonoperative segment/activities (e.g., using optical flow methods, a machine learning tracking system, etc.). A metrics generation component 905d may then be used to generate metrics, e.g., as disclosed herein (e.g., determining as metrics the temporal durations of each of the intervals and actions of FIGS. 5A-C and the metrics as discussed in FIG. 8). While metrics results may be presented directly to the reviewer in some embodiments, as described herein, some embodiments may instead provide some initial analytical assessment of the metric values, determining standard deviations relative to historical values, prioritizing greater tolerance departures for prioritized presentation to the reviewer, determining if metric values (e.g., motion) indicate that it would be desirable to perform a more refined analysis of the data (e.g., determining team member movement paths, object collision event detections, etc.), etc. Accordingly, a metrics analysis component 905e may then analyze the generated metrics, e.g., to determine outliers relative to historical patterns.

FIG. 9B is a schematic block diagram depicting elements in a more detailed example processing flow than the flow depicted in FIG. 9A, as may be implemented in some embodiments. One will appreciate that each depicted component may be logic or may be one or more machine learning systems, as discussed in greater detail herein. The computer system 910b may receive the theater wide sensor data 910a and first perform the nonoperative period detection 910c (e.g., identifying the periods 310a, 310b, 310c, 310d, though some systems may be configured to only detect nonoperative periods of the character of periods 310b, and 310c). Once the portions of the theater-wide data corresponding to the nonoperative periods have been detected, the data may then be further segmented into corresponding action tasks or intervals (e.g., the intervals 550a-d and/or groupings 510, 515, 520, 525 and respective action tasks) at block 910d.

Using object detection (and in some embodiments, tracking) machine learning systems 910e, the system may perform object detection using machine learning methods, such as of equipment 910f or personnel 910h (ellipsis 910g indicating the possibility of other machine learning systems). In some embodiments, only personnel detection 910h is performed, as only the number of personnel and their motion are needed for the desired metrics. Motion detection component 910i may then analyze the objects detected at block 910e to determine their respective motions, e.g., using various machine learning methods, optical flow, combinations thereof, etc. disclosed herein.

Using the number of objects, detected motion, and determined interval durations, a metric generation system 910j may generate metrics (e.g., the interval durations may themselves serve as metrics, the values of FIG. 8 may be calculated, etc.). The metric values may then be analyzed via component 910k to determine, e.g., outliers and other deviations from historical data (e.g., previous iterations of the topology 450). The system may consider 915a, 915c historical sensor data 915e and historical metrics data 915f when performing the historical comparison at block 910k (e.g., clustering historical metric values around efficient and inefficient nodes, then assessing the newly arrived data's distance to these nodes). In this manner, the system may infer that entire teams, groups of members, or individual members performed subpar compared to historical metrics data for similar roles, team member compositions, or individual team members. Conversely, the processed and raw theater-wide sensor data may be provided 915b to the historical data storage 915e for use in future analysis. Similarly, the metrics results and outlier determinations may be recorded 915d in the historical metrics database 915f for future reference.

The results of the analysis may then be presented via component 910l (e.g., sent over a network to one or more of applications 550f) for presentation to the reviewer. For example, application algorithms may consume the determined metrics and nonoperative data and propose customized actionable coaching for each individual in the team, as well as the team as a whole, based upon metrics analysis results (though such coaching or feedback may first be determined on the computer system 910b in some embodiments). Example recommendations include, e.g.: changes in the OR layout at various points in time, changes in OR scheduling, changes in communication systems between team members, changes in numbers of staff involved in various tasks, etc. In some embodiments, such coaching and feedback may be generated by comparing the metric values to a finite corpus of known inefficient patterns (or conversely, known efficient patterns) and corresponding remediations to be proposed (e.g., slow port placement and excess headcount may be correlated with an inefficiency resolved by reducing head count for that task).

For further clarity, FIG. 9C is a flow diagram illustrating various operations in an example overall process 920 for analyzing theater-wide data. At block 920a, the computer system may receive the theater-wide sensor data for the theater to be examined. At block 920b, the system may perform pre-processing on the data, e.g., reconciling theaterwide data to a common format, as when fisheye and rectilinear sensor data are both to be processed.

At block 920c, the system may perform operative and nonoperative period recognitions, e.g., identifying each of the segments 310a-d and 315a-c from the raw theater wide sensor data. In some embodiments, such divisions may be recognized, or verified, via ancillary data, e.g., console data, instrument kinematics data, etc. (which may, e.g., be active only during operative periods).

The system may then iterate over the detected nonoperative periods (e.g., periods 310a, 310b) at blocks 920d and 925a. In some embodiments, operative periods may also be included in the iteration, e.g., to determine metric values that may inform the analysis of the nonoperative segments, though many embodiments will consider only the nonoperative periods. For each period, the system may identify the relevant tasks and intervals at block 925b, e.g., the intervals, groups, and actions of FIGS. 5A-C.

At blocks 925c and 925e, the system may iterate over the corresponding portions of the theater data for the respectively identified tasks and intervals, performing object detections at block 925f, motion detection at block 925g, and corresponding metrics generation at block 925h. In some embodiments, at block 925f, only a number of personnel in the theater may be determined, without determining their roles or identities. Again, the metrics may thus be generated at the action task level, as well as at the other intervals described in FIGS. 5A-C. In alternative embodiments, the metrics may simply be determined for the nonoperative period (e.g., where the duration of the intervals 550a-e are the only metrics to be determined).

After all the relevant tasks and intervals have been considered for the current period at block 925c, then the system may create any additional metric values (e.g., metrics including the values determined at block 925h across multiple tasks as their component values) at block 925d. Once all the periods have been considered at block 920d the system may perform holistic metrics generation at block 930a (e.g., metrics whose component values depend upon the period metrics of block 925d and block 925h, such as certain composite metrics described herein).

At block 930b, the system may analyze the metrics generated at blocks 930a, 925d, and at block 925h. As discussed, many metrics (possibly at each of blocks 930a, 925h, and 925d) will consider historical values, e.g., to normalize the specific values here, in their generation. Similarly, at block 930b the system may determine outliers as described in greater detail herein, by considering the metrics results in connection with historical values. Finally, at block 930c, the system may publish its analysis for use, e.g., in applications 450f.

### Example Nonoperative Theater-Wide Data Processing-Nonoperative Data Recognition

One will appreciate a number of systems and methods sufficient for performing the operative/nonoperative period detection of components 905a or 910c and activity/task/interval segmentation of block 910d (e.g., identifying the actions, tasks, or intervals of FIGS. 5A-C). Indeed, as mentioned, in some embodiments, alternative signals than the theater-wide data or monitoring of gross-signals in the theater-wide data may suffice for distinguishing periods 310a-d from periods 315a-d. For example, in some embodiments, a team member may provide explicit notification. Similarly, the absence of kinematics and system events data from robotic surgical systemics consoles or instruments may indicate a prolonged separation between the surgeon and patient or between a robotic platform and the patient, which may suffice to indicate that an inter-surgical nonoperative period has begun (or provide verification of a machine learning system's parallel determination).

However, some embodiments consider instead, or in addition, employing machine learning systems for performing the nonoperative period detection. For example, some embodiments employ spatiotemporal model architectures, e.g., like a transformer architecture such as that described in Bertasius, Gedas, Heng Wang, and Lorenzo Torresani. “Is Space-Time Attention All You Need for Video Understanding?” arXiv™ preprint arXiv™: 2102.05095 (2021). Such approaches may also be especially useful for automatic activity detection from long sequences of theater-wide sensor data. The spatial segment transformer architecture may be designed to learn features from frames of theater-wide data (e.g., visual image video data, depth frame video data, visual image and depth frame video data, etc.). The time segment may be based upon a gated recurrent unit (GRU) method and designed to learn the sequence of actions in a long video and may, e.g., be trained in a fully supervised manner (again, where data labelling may be assisted by the activation of surgical instrument data). For example, OR theater-wide data may be first annotated by a human expert to create ground truth labels and then fed to the model for supervised training.

Some embodiments may employ a two-stage model training strategy: first training the back-bone transformer model to extract features and then training the temporal model to learn a sequence. Input to the model training may be long sequences of theater-wide data (e.g., many hours of visual image video) with output time-stamps for each segment (e.g., the nonoperative segments) or activity (e.g., intervals and tasks of FIGS. 5A-C) of interest. One will appreciate that some models may operate on individual visual images, individual depth frames, groups of image frames (e.g., segments of video), groups of depth frames (e.g., segments of depth frame video), combinations of visual video and depth video, etc.

As another example, FIG. 10 is a flow diagram illustrating various operations in an example process 1005 for performing nonoperative period detection in some embodiments. Specifically, as the number of theater-wide sensors may change across theaters, or across time in the same theater, it may be undesirable to invest in training a machine learning system configured to receive only a specific number of theater-wide data inputs. Thus, in these embodiments, where the classifier is not configured to consider the theater-wide sensor data from all the available streams at once, the system may instead consider the streams individually, or in smaller groups, and then analyze the collective results, e.g., in combination with smoothing operations, so as to assign a categorization to the segment under consideration.

For example, after receiving the theater-wide data at block 1005a (e.g., all of three streams 325a-c, 330a-c, and 335a-c) the system may iterate over the data in intervals at blocks 1005b and 1005c. For example, the system may consider the streams in successive segments (e.g., 30 second, one, or two minute intervals), though the data therein may be down sampled depending upon the framerate of its acquisition. For each interval of data, the system may iterate over the portion of the interval data associated with the respective sensor's streams at blocks 1010a and 1010b (e.g., each of streams 325a-c, 330a-e, and 335a-e or groups thereof, possibly considering the same stream more than once in different groupings). For each stream, the system may determine the classification results at block 1010c as pertaining to an operative or nonoperative interval. After all the streams have been considered, at block 1010d, the system may consider the final classification of the interval. For example, the system may take a majority vote of the individual stream classifications of block 1010c, resolving ties and smoothing the results based upon continuity with previous (and possibly subsequently determined) classifications.

After all the theater-wide data has been considered at block 1005b, then at block 1015a the system may consolidate the classification results (e.g., performing smoothing and continuity harmonization for all the data, analogous to that discussed with respect to block 1010d, but here for larger smoothing windows, e.g., one to two hours). At block 1015b, the system may perform any supplemental data verification before publishing the results. For example, if supplemental data indicates time intervals with known classifications, the classification assignments may be hardcoded for these true positives and the smoothing rerun.

### Example Nonoperative Theater-Wide Data Processing-Object Recognition

Like nonoperative and operative theater-wide data segmentation, one will likewise appreciate a number of ways for performing object detection (e.g., at block 905b or component 910c). Again, in some embodiments, object detection includes merely a number of personnel count, and so a You Only Look Once (YOLO) style network (e.g., as described in Redmon, Joseph, et al. “You Only Look Once: Unified, Realtime Object Detection.” arXiv™ preprint arXiv™: 1506.02640 (2015)), perhaps applied iteratively, may suffice. However, some embodiments consider using groups of visual images or depth frames. For example, some embodiments employ a transformer based spatial model to process frames of the theater-wide data, detecting all humans present and reporting the number. An example of such architecture is described in Carion, Nicolas, et al. “End-to-End Object Detection with Transformers.” arXiv™ preprint arXiv™: 2005.12872 (2020).

To clarify this specific approach, FIG. 11A is a schematic block diagram illustrating an example information processing flow as may be used for performing object detection in connection with some embodiments. Given a visual or depth frame image 1105f, the system may present the image's raw pixel or depth values to a convolutional network 1105a trained to produce image features 1105b. These features may in turn be provided to a transformer encoder-decoder 1105c and the bipartite matching loss 1105d used to make predictions 1105e for the location and number of objects (e.g., personnel or equipment) in the image, reflected here by bounding boxes within the augmented image 1105g (one will appreciate that an actual augmented image may not be produced by the system, but rather, only indications of the object locations and, in some embodiments, of the type of object found therein).

FIG. 11B is a flow diagram illustrating various operations in an example process 1100 for performing object detection as may be used in connection with some embodiments. At block 1110a, the system may receive the theater-wide data (visual image data, depth data, etc.). At blocks 1110b, and 1110c, as in the process 1005, the system may iterate over the nonoperative periods, considering the data in discrete, successive intervals (as mentioned, in some embodiments the operative periods may be considered as well, e.g., to verify continuity with the object detections and recognitions at the beginnings or ends of the nonoperative periods).

At blocks 1110d and 1115a the system may consider groups of theater-wide data. For example, some embodiments may consider every moment of data capture, whereas other embodiments may consider every other capture or captures at intervals, since some theater sensors may employ high data acquisition rates (indeed, not all sensors in the theater may apply a same rate and so normalization may be applied so as to consolidate the data). For such high rates, it may not be reasonable to interpolate object locations between data captures if the data capture rate is sufficiently larger than the movement speeds of objects in the theater. Similarly, some theater sensor's data captures may not be perfectly synchronized, or may capture data at different rates, obligating the system to interpolate or to select data captures sufficiently corresponding in time so as to perform detection and metrics calculations.

At blocks 1115b and 1115c, the system may consider the data in the separate theater-wide sensor data streams and perform object detection at block 1115d, e.g., as described above with respect to FIG. 11A, or using a YOLO network, etc. After object detection has been performed for each stream for the group under consideration, the system may perform post-processing at block 1115e. For example, if the relative poses of the theater-wide sensors are known within the theater, then their respective object detections may be reconciled to better confirm the location of the object in a three-dimensional representation such as a three-dimensional point cloud. Similarly, the relative data captures may be used to verify one another's determinations and to resolve occlusions based upon temporal continuity (e.g., as when a team member occludes one senor's perspective, but not another sensor's).

After all of the temporal groups have been considered at block 1110d, then at block 1110c, additional verification may be performed, e.g., using temporal information from across the intervals of block 1110d to reconcile occlusions and lacuna in the object detections of block 1115d. Once all the nonoperative periods of interest have been considered at block 1110b, at block 1120a, the system may perform holistic post-processing and verification in-filling. For example, knowledge regarding object presence between periods or based upon a type of theater or operation may inform the expected numbers and relative locations of objects to be recognized. To this end, even though some embodiments may be interested in analyzing nonoperative periods exclusively, the beginning and end of operative periods may help inform or verify the nonoperative period object detections, and may be considered. For example, if four personnel are consistently recognized throughout an operative period, then the system should expect to identify four personnel at the end of the preceding, and the beginning of the succeeding, nonoperative periods.

### Example Nonoperative Theater-Wide Data Processing-Object Tracking

As with segmentation of the raw data into nonoperative periods (e.g., as performed by nonoperative period detection component 910c), and the detection of objects, such as personnel, within those periods (e.g., via component 910c), one will appreciate a number of ways to perform tracking and motion detection. For example, object detection, as described, e.g., in FIG. 11B, in combination with optical flow analysis (with complementary stream perspectives resolving ambiguities) may readily be used to recognize each particular object's movement throughout the theater. As another example, some embodiments may employ multi-object machine learning tracking algorithms, which involve detecting and tracking multiple objects within a sequence of theater-wide data. These approaches may identify and locate objects of interest in each frame and then associate those objects across frames to keep track of their movements over time. For example, some embodiments may use an implementation analogous to that described in Meinhardt, Tim, et al. “TrackFormer: Multi-Object Tracking with Transformers.” arXiv™ preprint arXiv™: 2101.02702 (2021).

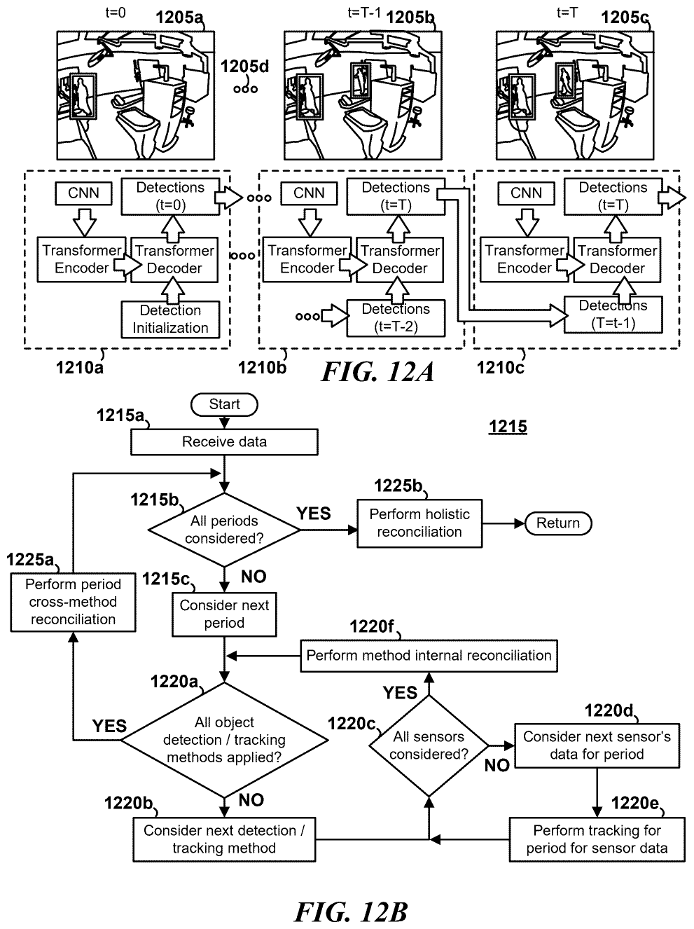

As an example in accordance with the approach of Meinhardt, et al., FIG. 12A is schematic block diagram illustrating an example tracking information processing flow as may be used in connection with some embodiments. In a first visual image or depth frame 1205a, the system may apply a tracking framework collection 1210a of convolution neural network, transformer encoders and decoders, and initial object detection (e.g., with the assistance of the object detection method of FIG. 11A). Iterative application 1210b and 1210c of the tracking framework to subsequent images or frames 1205b and 1205c may produce object detections, such as personnel, with a record of the positions across the frames 1205a, 1205b, 1205c (ellipsis 1205d reflect the presence of intervening frames and tracking recognitions).

FIG. 12B is flow diagram illustrating various operations in an example process 1215 for performing object tracking as may be used in connection with some embodiments. At block 1215a, the system may receive the theater-wide data, e.g., following nonoperative period identification. At blocks 1215b and 1215c the system may iterate over the nonoperative periods and for each period, iterate over the contemplated detection and tracking methods at blocks 1220a and 1220b. For each method, the sensor data streams may be considered in turn at blocks 1220c and 1220d, performing the applicable detection and tracking method at block 1220e (one will appreciate that alternatively, in some embodiments, the streams may be first integrated before applying the object detection and tracking systems, as when simultaneously acquired depth frames from multiple sensors are consolidated into a single virtual model). As mentioned, some methods may benefit from considering temporal and spatial continuity across the theater-wide sensors, and so reconciliation methods for the particular tracking application may be applied at block 1220f.

Similarly, reconciliation between the tracking methods' findings across the period may be performed at block 1225a. For example, determined locations for objects found by the various methods may be averaged. Similarly, the number of objects may be determined by taking a majority vote among the methods, possibly weighted by uncertainty or confidence values associated with the methods. Similarly, after all the nonoperative periods have been considered, the system may perform holistic reconciliation at block 1225b, e.g., ensuring that the initial and final object counts and locations agree with those of neighboring periods or action groups.

As one will note when comparing FIG. 12B and FIG. 9C, object detection, tracking, or motion detection may be performed at the period level (and then associated with tasks/actions/intervals for metrics calculation if desired) or may be performed after the actions, tasks, or intervals have been identified, and upon corresponding data specifically.

### Example Nonoperative Theater-Wide Data Processing-Motion Assessment

While some tracking systems may readily facilitate motion analysis at motion detection component 910i, some embodiments may alternatively, or in parallel, perform motion detection and analysis using visual image and depth frame data. In some embodiments, simply the amount of motion (in magnitude, regardless of its direction component) within the theater in three-dimensional space of any objects, or of only objects of interest, may be useful for determining meaningful metrics during nonoperative periods. However, more refined motion analysis may facilitate more refined inquiries, such as team member path analysis, collision detection, etc.

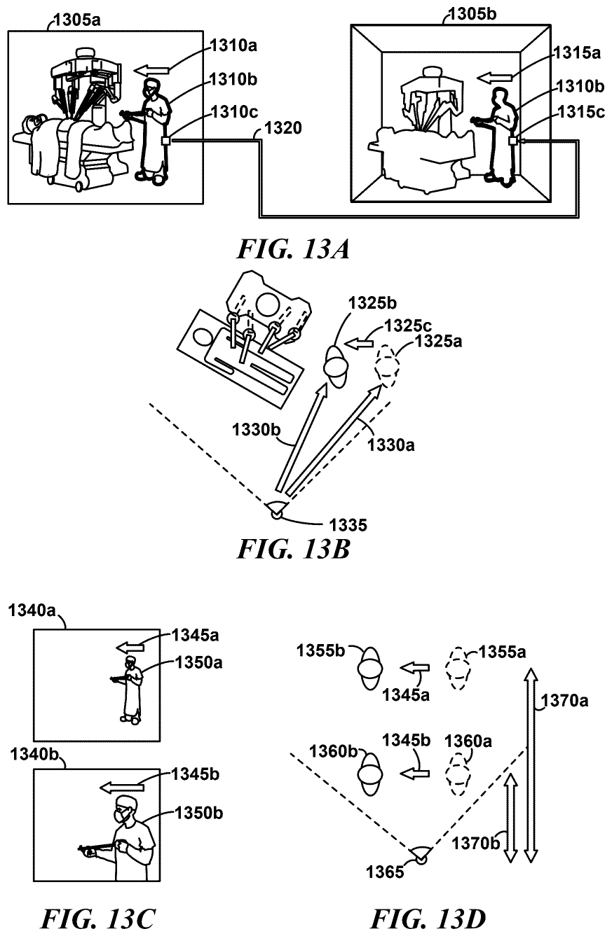

As an example optical-flow based motion assessment, FIG. 13A is a schematic visual image 1305a and depth frame 1305b theater-wide data pair, with an indication of the optical-flow derived correspondence as may be used in some embodiments. Specifically, the data processing system may review sequences of visual image data to detect optical flow. Here, the system has detected that the team member 1310b is moving from the right to the left of the image as indicated by arrow 1310a and by the pixel border around the pixels having optical flow around team member 1310b.

While some embodiments may consider motion based upon the optical flow from visual images alone, it may sometimes be desirable to “standardize” the motion. Specifically, turning to FIG. 13C, movement 1345a far from the camera, as shown in image 1340a may result in a smaller number of pixels (the pixels depicting the member 1350a) being associated with the optical flow. Conversely, as shown in image 1340b, when the team member 1350b is very close to the sensor, their motion 1345b may result in an optical flow affecting many more pixels.

Rather than allow the number of visual image pixels involved in the flow to affect the motion determination, some embodiments may standardize the motion associated with the optical flow to three-dimensional space. That is, with reference to FIG. 1 three-dimensional, the motions 1345a and 1345b may be the same in magnitude in three-dimensional space, as the team members move from locations 1355a, 1360a to locations 1355b, 1360b, respectively. While the locations 1360a-b are a smaller distance 1370b from the sensor 1365 than the distance 1370a from the sensor 1365 to the locations 1355a-b, some embodiments may seek to identify the same amount of motion 1345a, 1345b in each instance. Specifically, downstream metrics may treat the speed of the motions 1345a, 1345b equally, regardless of their distance from the capturing sensor.

To accomplish this, returning to FIG. 13A, for each portion of the visual image 1305a associated with the optical flow, the system may consider the corresponding portions of the simultaneously acquired depth image 1305b, here, where the team member 1310b and their motion, indicated by arrow 1315a, will also be manifest. That is, in this example the pixels 1310c associated with the optical flow may correspond 1320 to the depth values 1315c. By considering these depth values 1320, the system may infer the distance to the object precipitating the optical flow (e.g., one of distances 1370b and 1370a). That is, with reference to FIG. 13B, the system may be able to infer the “standardized” motion 1325c in three-dimensional space for the object moving from position 1325a to position 1325b, once the distances 1330a and 1330b from the capturing sensor 1335 have been inferred from the depth data. In some embodiments, in lieu of first detecting optical flow in the two-dimensional visual image, optical flow in the three-dimensional depth data may instead be used and the standardized motion determined mutatis mutandis.

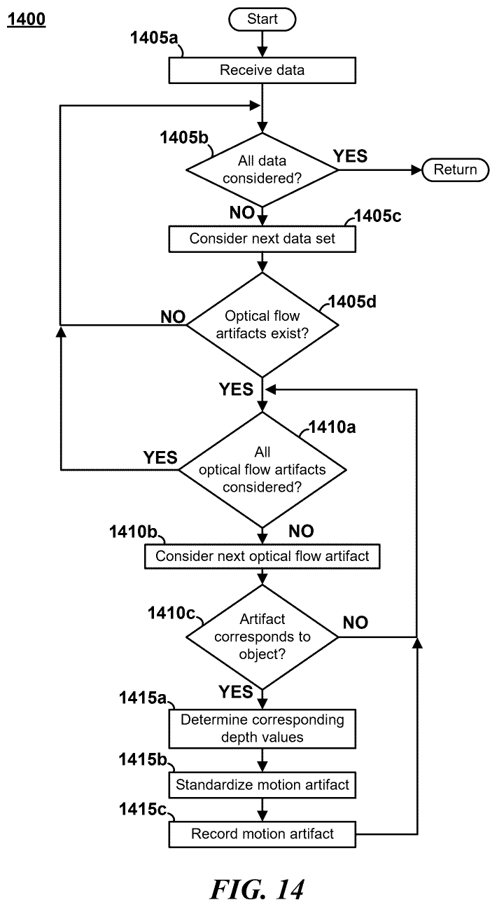

FIG. 14 is a flow diagram illustrating various operations in an example process 1400 for performing motion analysis from theater-wide data, as may be applied in some embodiments. At blocks 1405b and 1405c, the system may iterate over the theater-wide data received at block 1405a. For example, theater-wide data may be down sampled and considered in discrete data sets of temporally successive visual image and depth frame pairs. Where one or more optical flow artifacts (contiguous regions with optical flow above a threshold are detected in either the visual images or the depth frames) are detected within the data set at block 1405d, the system may iterate over the artifacts at blocks 1410a and 1410b. Many artifacts may not correspond to objects of interest for preparing metrics. For example, incidental motion of some equipment, adjustment of some lights, opening of some doors, etc., may not be relevant to the downstream analysis. Accordingly, at block 1410c, the system may verify that the artifact is associated with one or more of the objects of interest (e.g., the personnel or equipment detected using the methods disclosed herein via the machine learning systems of component 910c, e.g., including the systems and methods of FIGS. 11A-B and 12A-B), for example, pixels corresponding to the optical flow may be compared with pixels identified in, e.g., a YOLO network object detection. In some cases, a single optical flow artifact may be associated with more than one object, e.g., when one moving object occludes another moving object. Assessment of the corresponding depth values may reveal the identities of the respective objects appearing in the artifact or at least their respective locations and trajectories.

Thus, where the artifact corresponds to an object of interest (e.g., team personnel), then at block 1415a, the system may determine the corresponding depth values and may standardize the detected motion at block 1415b to be in three-dimensional space (e.g., the same motion value regardless of the distance from the sensor) rather than in the two-dimensional plane of a visual image optical flow, e.g., using the techniques discussed herein with respect to FIGS. 13A-D. The resulting motion may then be recorded at block 1415c for use in subsequent metrics calculation as discussed in greater detail herein.

### Example Nonoperative Theater-Wide Metrics Analysis-Outlier Detection

Following metrics generation (e.g., at metric generation system 910j) some embodiments may seek to recognize outlier behavior (e.g., at metric analysis system 910k) to detect outliers in each team/operating room/hospital/etc. based upon the above metrics, including the durations of the actions and intervals in FIGS. 5A-C, the numbers of people involved in each theater and the amount of motion observed, etc. For example, FIG. 15 is flow diagram illustrating various operations in an example process 1500 for outlier analysis based upon the determined metric values, as may be implemented in some embodiments.

At block 1505a, the system may acquire historical datasets, e.g., for use with metrics having component values (such as normalizations) based upon historical data. At block 1505b, the system may determine metrics results for nonoperative period as a whole (e.g., cumulative motion within the period, regardless of whether it occurred in association with any particular task or interval). At block 1505c, the system may determine metrics results for specific tasks and intervals within each of the nonoperative segments (e.g., the durations of actions and intervals in FIGS. 5A-C). At block 1505d, the system may then determine composite metric values from the previous of the determined metrics (e.g., the ORA score 830 discussed in FIG. 8).

At block 1505e, clusters of metric values corresponding to patterns of inefficient or efficient nonoperative theater states, as well as clusters of metric values corresponding to patterns of efficient or positive nonoperative theater states, may be included in the historical data of block 1505a. Such clusters may be used both to find metric scores, and patterns of metrics scores, distance from ideal clusters and distance from undesirable clusters (e.g., where the distance is the Euclidean distance and each metric of a group is considered as a separate dimension).

Thus, the system may the iterate over the metrics individually, or in groups, at blocks 1510a and 1510b to determine if the metrics or groups exceed a tolerance at block 1510c relative to the historical data clusters (naturally, the nature of the tolerance may change with each expected grouping and may be based upon a historical benchmark, such as one or more standard deviations from a median or mean). Where such tolerance is exceeded (e.g., metric values or groups of metric values are either too close to inefficient clusters or too far from efficient clusters), the system may document the departure at block 1510d for future use in coaching and feedback as described herein.

For clarity, as mentioned, the cluster may occur in an N dimensional space where there are N respective metrics considered in the group (though alternative spaces and surfaces for comparing metric values may also be used). Such an algorithm may be applied to detect outliers for each team/operating room/hospital based upon the above metrics. Cluster algorithms (e.g., based upon K-means, using machine learning classifiers, etc.) may both reveal groupings and identify outliers, the former for recognizing common inefficient/efficient patterns in the values, and the latter for recognizing, e.g., departures from ideal performances or acceptable avoidance of undesirable states.

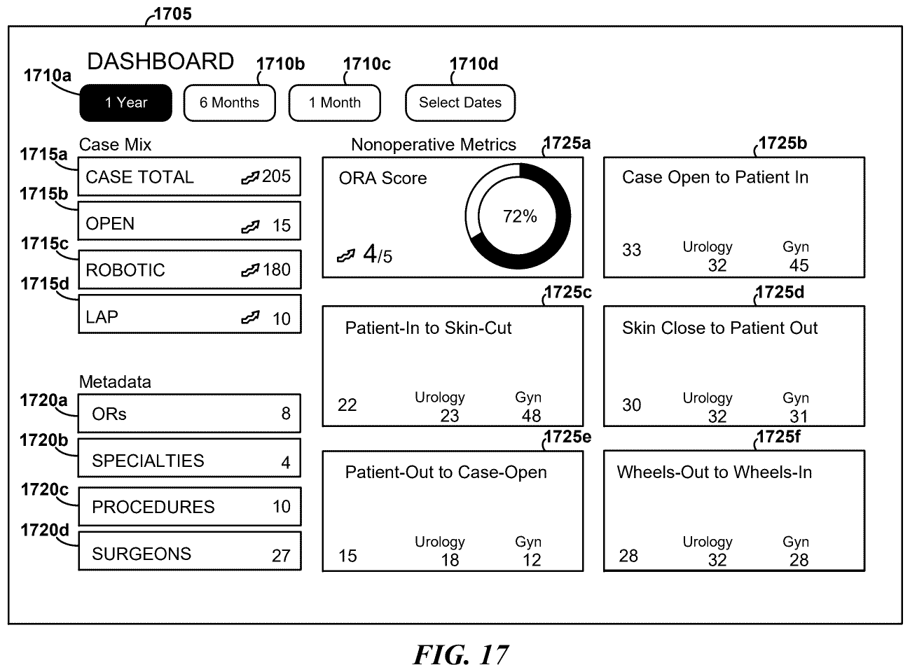

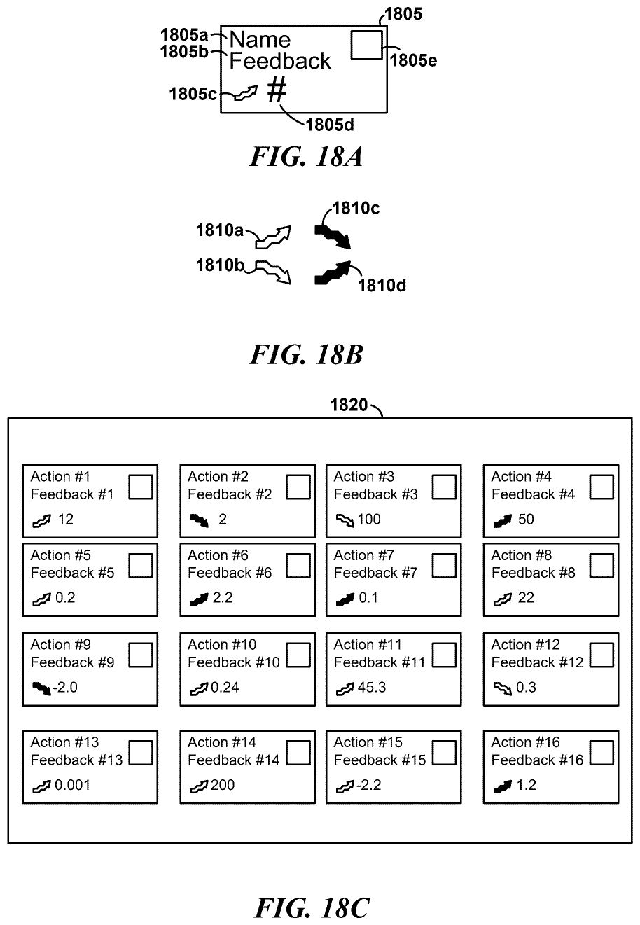

Thus the system may determine whether the metrics individually, or in groups, are associated (e.g., within a threshold distance of, such as the cluster's standard deviation, larges principal component, etc.) with an inefficient, or efficient, cluster at block 1515a, and if so, document the cluster for future coaching and feedback at block 1515b. For example, raw metric values, composite metric values, outliers, distances to or from clusters, correlated remediations, etc., may be presented in a GUI interface, e.g., as will be described herein with respect to FIG. 17 or 18A-C.

### Example Nonoperative Data Analysis-Coaching

Following outlier detection and clustering, in some embodiments, the system may also seek to consolidate the results into a form suitable for use by feedback and coaching (e.g., by the applications 550f). For example, remediating actions may already be known for tolerance breaches (e.g., at block 1510c) or nearness to adverse metrics clusters (e.g., at block 1515a). Here, coaching may, e.g., simply include the known remediation when reporting the breach or clustering association.

Some embodiments may recognize higher level associations in the metric values, from which remediations may be proposed. For example, after considering a new dataset from a theater in a previously unconsidered hospital, various embodiments may determine that a specific surgical specialty (e.g., Urology) in that theater, possesses a large standard deviation in its nonoperative time metrics. Various algorithms disclosed herein may consume such large standard deviations, other data points, and historical data and suggest corrective action regarding with scheduling or staffing model. For example, a regression model may be used that employs historical data to infer potential solutions based upon the data distribution.

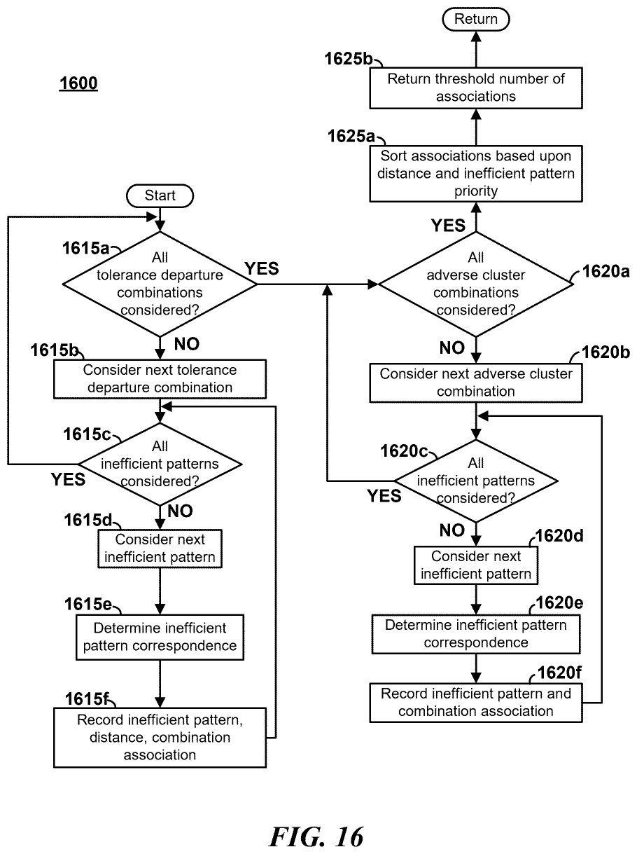

As another example, FIG. 16 is flow diagram illustrating various operations in an example process 1600 for providing coaching feedback based upon the determined metric values, as may be implemented in some embodiments. While focusing on relations between metric values and adverse/inefficient patterns in this example, one will appreciate variations that instead determine relations to desirable/efficient patterns (with corresponding remediations when the metrics depart too far from these preferred states). Similarly, in some embodiments, metrics and groups of metrics may be directly compared to known patterns without first identifying tolerance departures and cluster distances, as in the example process 1600.

Here, at blocks 1615a and 1615b, the system may iterate over all the previously identified tolerance departures (e.g., as determined at block 1510c) for the groupings of one or more metric results and consider whether they correspond with a known inefficient pattern at block 1615c (e.g., taking an inner product with the metric values with a known inefficient vector). For example, a protracted “case open to patient in” duration in combination with certain delay 810c and case volume 810a values, may, e.g., be indicative of a scheduling inefficiency where adjusting the scheduling regularly resolves the undesirable state. Note that the metric or metrics used for mapping to inefficient patterns for remediation may, or may not, be the same as the metric or metrics, which departed from the tolerance (e.g., at block 1615a) or approached the undesirable clustering (e.g., at block 1620a), e.g., the latter may instead indicate that the former may correspond to an inefficient pattern. For example, an outlier in one duration metric from FIG. 5A may imply an inefficient pattern derived from a combinations of metrics from FIG. 8.

Accordingly, the system may iterate through the possible inefficient patterns at blocks 1615c and 1615d to consider how the corresponding metric values resemble the inefficient pattern. For example, the Euclidean distance from the metrics to the pattern may be taken at block 1615e. At block 1615f, the system may record the similarity (e.g., the distance) between the inefficient pattern and the metrics group associated with the tolerance departure.

Similarly, following consideration of the tolerance departures, the system may consider metrics score combinations with clusters near adverse/inefficient events (e.g., as determined at block 1515a) at blocks 1620a and 1620b. As was done previously, the system may iterate over the possible known inefficient patterns at blocks 1620c and 1620d, again determining the inefficient pattern correspondence to the respective metric values (which may or may not be the same group of metric values identified in the cluster association of block 1620a) at block 1620c (again, e.g., the Euclidean or other appropriate similarity metric) and recording the degree of correspondence at block 1620f.

Based upon the distances and correspondences determined at blocks 1615e and 1620e, respectively, the system may determine a priority ordering for the detected inefficient patterns at block 1625a. At block 1625b, the system may return the most significant threshold number of inefficient pattern associations. For example, each inefficient pattern may be associated with a priority (e.g., high priority modes may be those with a potential for causing a downstream cascade of inefficiencies, patient harm, damage to equipment, etc., whereas lower priority modes may simply lead to temporal delays) and presented accordingly to reviewers. Consequently, each association may be scored as a weighted sum of a similarity between the score metric values and metric values associated with inefficient pattern and then weighted by the severity/priority of the inefficient pattern. In this manner, the most significant of the possible failures may be identified and returned first to the reviewer. The iterative nature of topology 450 may facilitate reconsideration and reweighting of the priorities for process 1600 as reviewers observe the impact of the proposed feedback over time. Similarly, the iterations may provide opportunities to identify additional remediation and inefficient pattern correspondences.

### Example GUI Nonoperative Metrics Analysis Feedback Elements

Presentation of the analysis results, e.g., at block 910l, may take a variety of forms in various embodiments. For example, FIG. 17 is a schematic representation of GUI elements in a quick review dashboard interface for nonoperative metrics review as may be implemented in some embodiments. In this example GUI 1705, selectors 1710a-d are provided for the user to select the temporal range of nonoperative period performance data that they wish to analyze. In this example, the user has selected to review the data captured during the past year. Following such a temporal selection, a “Nonoperative Metrics” region, a “Case Mix” region, and a “Metadata” region may be populated with values corresponding to the nonoperative periods for the selected range of data.

The “Case Mix” region may provide a general description of the data filtered from the temporal selection. Here, for example, there are 205 total cases (nonoperative periods) under consideration as indicated by label 1715a. A decomposition of those 205 cases is then provided by type of surgery via labels 1715b-d (specifically, that of the 205 nonoperative periods, 15 were associated with preparation for open surgeries, 180 with preparation for a robotic surgery, and 10 with preparation for a laparoscopic surgery). The nonoperative periods under consideration may be those occurring before and after the 205 surgeries, only those before, or only those after, etc., depending upon the user's selection.

The “Metadata” region may likewise be populated with various parameters describing the selected data, such as the number of ORs involved (8 per label 1720a), the number of specialties (4 per label 1720b), the number of procedure types (10 per label 1720c) and the number of different surgeons involved in the surgeries (27 per label 1720d).

Within the “Nonoperative Metrics” region, a holistic composite score, such as an ORA score, may be presented in region 1725a using the methods described herein (e.g., as described with respect to FIG. 8). Regions 1725b-f may show corresponding statistics for the intervals of FIG. 5A, here, values for various intervals of FIG. 5A.

Some embodiments may also present scoring metrics results comprehensively, e.g., to allow reviewers to quickly scan the feedback and to identify effective and ineffective aspects of the nonoperative theater performance. For example, FIG. 18A is a schematic representation of a GUI element 1805 as may be used for global quick review feedback in some embodiments. Specifically, individual metrics score values, composite metric scores, departures from tolerances, nearness to desirable or undesirable clustering, etc. may be indicated in a numerical region 1805d. The name of the metrics, etc., may be indicated in the name region 1805a and a desired feedback in region 1805b. A quick review icon 1805e may also be included to facilitate ready identification of the nature of the numerical feedback. A quality relation arrow region 1805c may be used to indicate whether the numerical value in region 1805d is above or below an operational point or tolerance, or trending upward or downward over time, and whether this is construed as indicative of improving or decreasing efficiency.

Specifically, FIG. 18B is a schematic representation of arrow elements as may be used in the quality relation arrow region 1805c of FIG. 18A in some embodiments. The arrow may be, e.g., color-coded to indicate whether the value is efficient (e.g., green) or inefficient (e.g., red). Thus, a rising arrow 1810a may indicate that the value in region 1805d is above a lower bound (e.g., when an idle time following successful completion of a task has increased above a historical average). Similarly, the falling arrow 1810b may indicate that the value in region 1805d is below an upper bound (e.g., when an preparation time has decreased below a historical average). Conversely, a falling arrow 1810c may indicate that the value in region 1805d is below a desired minimum value (e.g., when a number of personnel ready for a required step is below a historical average). Similarly, the rising arrow 1810d may indicate that the value in region 1805d is above a desired upper bound (e.g., when an preparation time has increased beyond a historical average).

By associating relational value both with the arrow direction and highlighting (such as by color, bolding, animation, etc.), reviewers may readily scan a large number of values and discern results indicating efficient or inefficient feedback. Highlighting may also take on a variety of degrees (e.g., alpha values, degree of bolding, frequency of an animation, etc.) to indicate a priority associated with an efficient or inefficient value. For example, FIG. 18C is a schematic representation of GUI elements in a quick review feedback interface 1820 as may be used in some embodiments. Here, the individual quick review feedbacks (instances of the element 1805) may be arranged in a grid and sized so that the reviewer may perceive multiple items at one time. Each element may be selectable, presenting details for the value determination, including, e.g., the corresponding historical data, theater-wide data, intermediate metrics calculation results, etc. One will appreciate that the figure is merely schematic and each “Action” or “Feedback” text may be replaced with one of the metrics described herein (e.g., a duration of intervals from FIGS. 5A-C) and remediations, respectively (though in some configurations the feedback may be omitted from all or some of the elements).

FIG. 19A is a plot of example analytic values as acquired in connection with a prototype implementation of an embodiment. Specifically, FIG. 19A shows results following processing for various of the intervals of FIG. 5A. Here, an outlier value 1905a clearly indicates a deviation in the “skin close to patient out” interval from the median duration of ˜10 minutes (taking instead approximately ˜260 minutes). FIG. 19B is similarly a plot of example operating room analytic values as acquired in connection with a prototype implementation of an embodiment. Here, standard deviation intervals may be shown to guide the reviewer in recognizing outlier values (e.g., whether they reflect a longer or shorter duration than the standard deviation interval).

FIG. 20A is a plot of example values as acquired in connection with a prototype implementation of an embodiment. Even without performing the outlier detection and inefficient pattern recognition methods disclosed herein, one can readily determine by inspection that various of the values are outliers. For example, within the “case open to patient in” interval, the cases 2005a-c clearly indicate outliers above the standard deviation. For the “patient in to skin cut” interval, the case 2010a is shorter than the standard deviation interval. For the “skin close to patient out” interval, the cases 2015a-b were outside the standard deviation for the selected historical cases. For the “patient out to case open” interval, the case 2020a lies far outside the standard deviation for the selected historical cases. For the “wheels out to wheels in” interval, the cases 2025a and 2025b lie outside the standard deviation for the selected historical cases. FIG. 20B is similarly a plot of example operating room analytic values as acquired in connection with a prototype implementation of an embodiment.

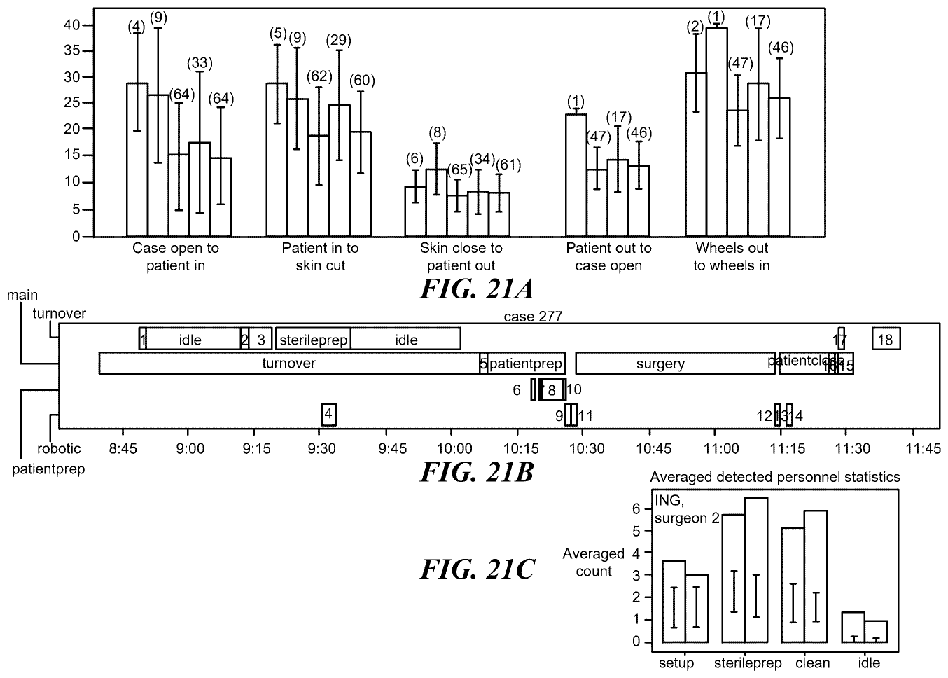

Similarly, FIG. 21A is a plot of example operating room analytic values as acquired in connection with an example prototype implementation of an embodiment. FIG. 21B is a plot of example operating room analytic values as acquired in connection with an example prototype implementation of an embodiment, in a horizontal format. FIG. 21C is also a plot of example operating room analytic values as acquired in connection with an example prototype implementation of an embodiment.

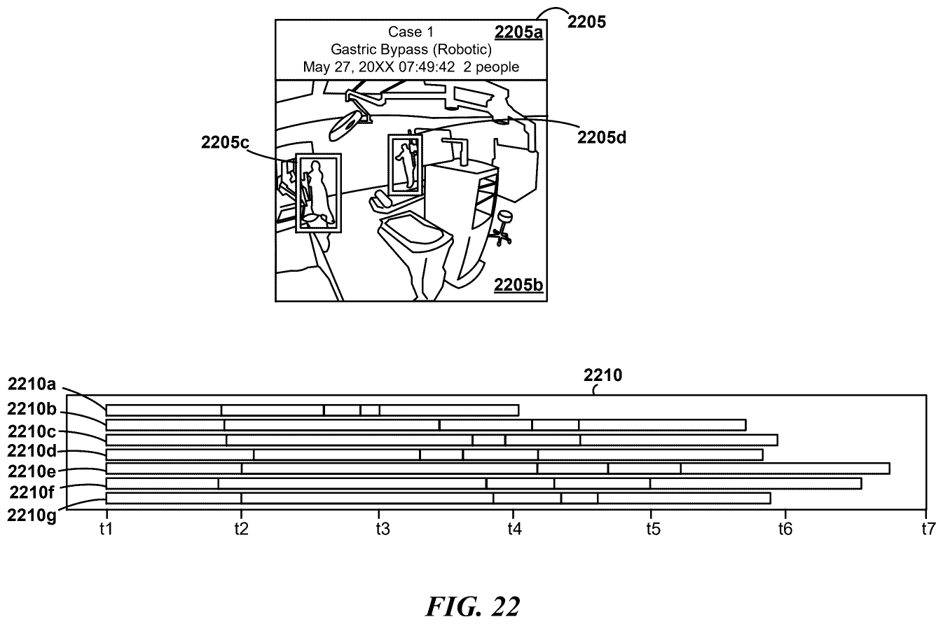

FIG. 22 is a schematic representation of example elements in a graphical user interface for providing metrics-derived feedback, as may be used in some embodiments. In this example, the interface includes two elements: a theater-wide sensor playback element 2205; and a consolidated timeline element 2210 depicting the durations of various intervals or segments within a plurality of nonoperative periods. For example, each of temporal interval breakdowns 2210a-g may indicate the durations of intervals 550a-d for ready comparison (though seven periods are shown in this example, one may readily envision variations with many more rows, as well as more instances of playback element 2205).

Within the theater-wide sensor playback element 2205 may be a metadata section 2205a indicating the identity of the case (“Case 1”), the state of the theater (though a surgical operation “Gastric Bypass”, is shown here, in anticipation of the upcoming surgery, the nonoperative actions and intervals of FIG. 5A-C may be shown here additionally or alternatively), the date and time of the data acquisition (“May 27, 20XX 07:49:42”) and the number of identified personnel (here “2” as determined, e.g., in accordance with component 910h and, e.g., the methods of FIGS. 11A-B). The theater-wide sensor playback element 2205 may also display the data from one or more theater-wide sensors in the playback section 2205b with bounding boxes (e.g., boxes 2205c and 2205d), overlays, outlines, or other suitable indications of the personnel detected. In the consolidated timeline element 2210 a plurality of temporal intervals 2210a-g may be rendered, indicating, e.g., a plurality of interval durations (e.g., the durations of intervals 550a-d for seven nonoperative periods). The playback in the region 2205 may correspond to a selection of one of the intervals in the temporal interval breakdowns 2210a-g (e.g., depicting corresponding theater-wide data playback for that interval). In this manner, the reviewer may readily perceive a corpus of results while simultaneously analyzing the state of a specific instance (e.g., as may have been called to the user's attention based upon, e.g., correspondingly determined metric values or pattern similarities).

### Metric Prediction Using Robotic System Data

In a medical environment analytics system as described herein with reference to FIGS. 1-22, the streams of multimodal data (e.g., theater-wide data) are collected using sensors (e.g., theater-wide sensors such as the sensors 170a, 170c, 220a, 220b, and 220c, exocentric sensors, egocentric sensors, etc.) and edge computing deployed within a medical environment. Authorizing, acquiring, installing, configuring, calibrating, and testing the sensors for a medical environment (e.g., an OR) may require allocation of resources to meet the hardware implementation costs for the medical environment analytics system. Hardware and software implementation costs for generating and providing the metrics can be significantly reduced by using the robotic system data to estimate or predict at least some of such metrics, given that a robotic system configured to output the robotic system data may already been acquired for the medical environment to perform robot-assisted surgery. In this scenario, hospital administrators, consultants, medical education institutions, and care teams can obtain valuable preliminary insight into medical environment schedules, procedures and guidelines, best practices, layouts, etc. via the metrics predicted using the robotic system data without first deploying the hardware (e.g., the theater-wide sensors, edge computing, etc.) and the software/firmware for the medical environment analytics system. Should the predicted metrics be deemed beneficial for improving the medical environment layouts, schedules, procedures and guidelines, best practices, etc., the medical environment analytics system can then be deployed to provide the more comprehensively metrics determined using the theater-wide sensors.

Certain advanced and sophisticated robotic surgical systems such as the da Vinci™ surgical systems (including both single-port and multiport systems) can automatically generate robotic system data during the course of their operations. Robotic system data is a stream of data indicative of an activation state (e.g., activated or deactivated) or a pose (e.g., a position and orientation) of a component of the robotic surgical system. The robotic system data can be detected using one or more sensors (e.g., camera, infrared sensor, ultrasonic sensors, etc.), actuators, interfaces, consoles, and so on that can output information used to detect such robotic system data. For instance, robotic system data can include one or more data streams including, for example, events data, kinematics data, force data, etc. The events data can indicate or otherwise correspond to events during operation of the robotic system. The kinematics data can indicate or otherwise correspond to kinematic data of one or more manipulators of the robotic system, one or more manipulator assemblies of the robotic system, and/or instruments supported by the robotic system. The force data can indicate or otherwise correspond to force exerted on or experienced by one or more instruments of the robotic system. In certain embodiments, at least some of the instruments supported by the robotic system may include force sensors (e.g., strain gauge sensors, etc.) configured to sense forces exerted on or experienced by a portion of the instruments. In other words, the robotic system data can capture all actions of a robotic system and interactions between the robotic system and a surgeon operating the robotic system in a medical procedure. In addition to assessing surgeon performance, the robotic system data can also be adapted for predicting a metric due to the correlations and correspondence between the robotic system data and efficiency-related data.

As used herein, efficiency-related data includes one or more of the theater-wide data, identified segments (e.g., periods, phases, and tasks) of a medical procedure, a timeline of the identified segments, metric values calculated for the segments or for the entirety of the medical procedure, and so on. The segments can be identified and the metrics can be calculated as described with reference to FIGS. 1-22. The theater-wide data captures a complete and accurate list of activities in the medical environment including information beyond the medical procedure and interactions with the robotic system. As described herein, the theater-wide data can be multimodal, privacy preserving, and agnostic to a procedure type or modality (e.g., robotic surgery, non-robotic surgery, etc.) of the medical procedure. For example, the theater-wide data can be used to determine the layout of medical environments, room layouts, staff experience, scheduling, and so on.

Examples of a nonoperative period include the nonoperative periods 310a, 310b, 310c, 310d. In some embodiments, the nonoperative periods can be detected at 910c and 920c. Examples of a task within a nonoperative period include the tasks 320a, 320b, 320c, 320d, 320f, and 320e. As described herein, two or more tasks can be grouped as a phase or a stage. Examples of a phase include post-surgery 520, turnover 525, pre-surgery 510, and surgery 515, and so on. Accordingly, the data streams such as the multimodal data, theater-wide data, depth data, video data, etc. can be segmented into a plurality of periods, including operative periods and nonoperative periods. Each nonoperative period includes at least one phase. Each phase includes at least one task. A period, phase, or task can be collectively referred to as a segment or time segment. A timeline (e.g., a timeline 2500) of a medical procedure includes segments identified for a medical procedure.

In some embodiments, a prediction system as described herein includes one or more machine learning models and/or functions that can predict one or more metrics, referred to as predicted metrics. Examples of predicted metrics include ORA metrics (e.g., ORA scores), efficiency or inefficiency metrics, and other metrics described herein. In other words, the predicted metrics can be the same types of metrics as those determined using the medical environment analytics system as described in FIGS. 1-22. In general, the prediction system can extract workflow patterns/signatures (e.g., features) from the robotic system data and predict corresponding metric values, leveraging the correlations between features of the efficiency-related data and features of the robotic system data. In some examples, console time, system setup time, draping time, intraoperative disruptions, and so on reflected in the robotic system data can be indicative of other inefficiencies in the non-operative portions of a medical procedure, which can be measured using the predicted metrics as described herein. In some examples, as a care team encounters challenges in using a robotic system (e.g., long surgeon head-out time), the care team may also encounter additional and corresponding challenges in room turn-over, room setup, idle times, and so on that can be reflected in the predicted metrics as a result.

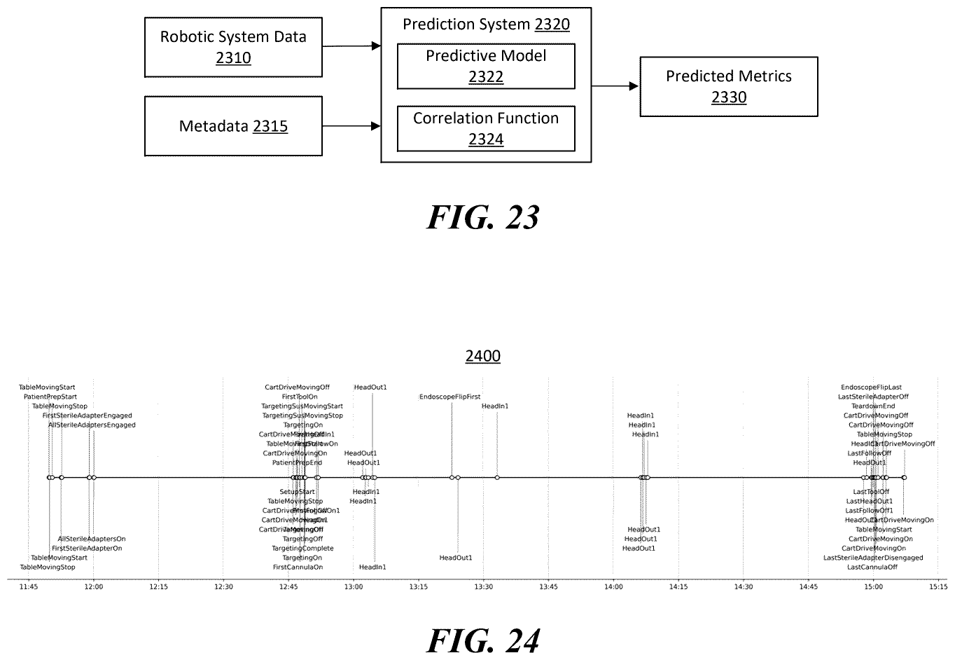

The embodiments disclosed herein are directed to systems, methods, apparatuses, and non-transitory computer-readable media for predicting metrics evaluating the efficiency and inefficiency of medical procedures and medical environments using robotic system data. FIG. 23 is a block diagram of an example prediction system 2320, according to some embodiments. As shown in FIG. 23, the prediction system 2320 can receive as an input robotic system data 2310 (e.g., first robotic system data) and output the predicted metrics 2330 (e.g., first metric values or range or values). In other words, the prediction system 2320, which is an artificial intelligence system and includes at least one machine learning model, can predict or estimate the predicted metrics 2330 using robotic system data 2310. During deployment, the prediction system 2320 can predict or estimate the predicted metrics 2330 in real time as the robotic system data 2310 is being collected during a medical procedure, to provide the predicted metrics 2330 for display during or after the medical procedure.

The prediction system 2320 can be implemented using one or more suitable computing systems, such as one or more computing systems 190a, 190b, 450b, and 3000. Code, instructions, data structures, weights, biases, parameters, and other information that define the prediction system 2320 can be stored in one or more memory systems such as one or more memory components 3015 and/or one or more storage systems 3025. In some examples, the processes of the prediction system 2320 such as predicting the metrics 2330 can be performed using one or more processors such as one or more processors 3010.

A robotic system (e.g., a robotic manipulator system, a robotic surgical system, etc.) can generate voluminous and detailed robotic system data 2310 during the course of performing a medical procedure in a medical environment. A robotic system can be any one of the robotic systems described herein, such as the system including the patient side cart 130, the surgeon console 155, etc. In some examples, two or more robotic systems can be present in a same medical environment to be used simultaneously and/or sequentially for a same medical procedure, and the robotic system data 2310 can include data from both or at least one of those robotic systems. In some examples, a robotic system can provide (e.g., send, communicate, or transfer via a suitable network) in real time the robotic system data 2310 to the prediction system 2320 directly or to an intermediate data collection and analysis system (e.g., the electronics/control console 145, the systems 190a, 190b, 450b, etc.) which in turn provides the robotic system data 2310 to the prediction system 2320. In some examples, a robotic system or the intermediate data collection and analysis system can provide the robotic system data 2310 to a suitable storage device or database for storage via a suitable network, where the prediction system 2320 can later retrieve the robotic system data 2310 for predicting the predicted metrics 2330.

In some implementations, the prediction system 2320 can receive as input the robotic system data 2310 from one or more robotic systems. In some examples, the robotic system data 2310 can be in natural language (e.g., a string of characters). The robotic system data 2320 includes kinematics data of each robotic system, system events data of each robotic system, input received by a console of each robotic system from a user, force data, and timestamps associated therewith. The robotic system data 2310 of a robotic system can be generated by the robotic system in its normal course of operations in the form of a robotic system log represented as timestamps in a timeline with a string or text characters defining the type of robotic system data 2310.

The kinematics data can indicate configurations of manipulators, manipulator assemblies, and actuators of the robotic system as well as the instruments attached or operatively coupled to the manipulators or manipulator assemblies over time throughout the medical procedure. Example kinematics data can include a pose of a manipulator, a manipulator assembly, and an instrument at various times throughout the medical procedure. For example, the kinematics data can be indicative of a pose of an actuator of a given arm (or an instrument attached thereto) of the robotic system at a given time or over a time interval. The force data can indicate or otherwise correspond to force exerted on or experienced by one or more instruments of the robotic system. In some examples, an instrument supported by the robotic system may include force sensors (e.g., strain gauge sensors, etc.) configured to sense forces exerted on or experienced by a portion of the instruments.

System events data can indicate system events of the robotic system. System events can specify activation state (e.g., activated or deactivated) of a component such as an actuator, manipulator, instrument, etc. Examples of system events include, for example, a docking event (e.g., in which manipulator arms are docked to cannulas inserted into a patient anatomy), operator (e.g., surgeon) head-in or head-out event (e.g., indicating a surgeon's head being present or absent at a viewer on a input or control console of the robotic system), an instrument attachment or removal event (e.g., indicating attachment or removal of an instrument, such as a medical instrument or an imaging instrument, on a manipulator of the robotic system, a tool exchange event), an instrument change event (e.g., indicating performance of an exchange of one instrument for another instrument for attachment on a manipulator on the robotic system), a draping-start event or a sterile adapter attachment event (e.g., which may indicate beginning of a sterile draping process), and the like. The robotic system can include one or more sensors (e.g., camera, infrared sensor, ultrasonic sensors, etc.), actuators, interfaces, consoles, that can output information used to detect such a system event. For example, the robotic system can include an interface configured to attach, communicate, control, and power an instrument, where such interface can output information indicating whether an instrument is attached. Some system events can be detected in response to user input by the surgeon in the surgeon console 155.

FIG. 24 is an example timeline 2400 (e.g., a system events timeline) including robotic system data (e.g., system events) over time, according to some embodiments. Each system event shown in the timeline 2400 can be described or logged as a string describing the system event, and has an associated timestamp and additional information. For example, for a system event such as an instrument-on event, the addition information can include an identity of the instrument. As shown, examples of the system events in the timeline 2400 include a head-in event (“HeadIn”), head-out event (“HeadOut”), a robot-in-follow event (“Targeting SusMovingStart”), a tool-on event (“FirstToolOn”), a tool-off event (“FirstToolOff”), first-cannula-on event (“FirstCannulaOn”), a last-cannula-off event (“LastCannulaOn”), a table-moving-start event (“TableMovingStart”), a table-moving-end event (“TableMovingEnd”), a cart-drive-enabled event (“CartDriveMovingOn”), a cart-drive-disabled event (“CartDriveMovingOff”), a first or all sterile-adapter-engaged event (“FirstSterileAdapterOn” or “AllSterileAdaptersOn”), a first or all sterile-adapter-disengaged event (“FirstSterileAdapterOff” or “AllSterileAdaptersOff”), and so on. Kinematics data can be likewise represented in a timeline, with a set of coordinates defining a pose of a component of the robotic system at a certain timestamp. Other examples of system events and kinematics data are likewise applicable. In some examples, both the robotic system data and the kinematics data can be included in a same timeline.

In some examples, the system event data can include a compound system event that is or corresponds to the time interval between the timestamps of two system events. In addition to single timestamps of system events, a time interval between two related system events can be effective predictors of the predicted metrics. In some examples, two timestamps that form a compound system event can be different timestamps from a same case or medical procedure. In some examples, two timestamps that form a compound system event can be the same or different timestamps from different cases or medical procedures. A compound system event can be highly correlated with a nonoperative metric or interval. For instance, the time interval between the timestamp of “last sterile adapter off” system event in one case to the timestamp of “first sterile adapter on” system event in a subsequent case can be useful in predicting a non-operative time between the two cases. In some examples, the time interval between “last sterile adapter off” system event in one case to “first sterile adapter on” system event in a subsequent case has significantly high correlation with the length for and the metric corresponding to “wheels out to wheels in” interval 550f. in another example, the time interval between the timestamp of “last follow off” system event (the robotic system detecting that the surgeon has exited control of the console) in one case to the timestamp of “first follow on” system event (the robotic system detecting that the surgeon has initiated control of the console) in a subsequent case can be useful in predicting a non-operative time between the two cases. In some examples, the time interval between “last follow off” system event in one case to “first follow on” system event in a subsequent case has significantly high correlation with the length for and the metric corresponding to “wheels out to wheels in” interval 550f. Other examples of correlation between compound system events and metrics for nonoperative intervals can be likewise implemented.

In some examples, the prediction system 2320 can receive metadata 2315 for the current medical procedure (e.g., the first medical procedure) as an input in addition to the robotic system data 2310 for the current medical procedure, to determine the predicted metrics 2330. As described in further details herein, the metadata 2315 can provide context for the current medical procedure in relation to the robotic system data 2310.

The predicted metrics 2330 (e.g., a metric value or a range of metric values) can be the same types of metrics determined via the workflow analytics 450e using the multimodal data (e.g., the theater-wide data), except that the predicted metrics 2330 are estimated or predicted by the prediction system 2320 rather than being determined using the methods described in FIGS. 1-22. Thus, the predicted metrics 2330 (e.g., a first metric value or a first range of metric values) are also indicative of the spatial and temporal efficiencies and inefficiencies of the medical procedure for which the robotic system data 2310 is collected. Example types of the predicted metrics 2330 include the metrics 805a, 805b, 805c, 810a, 810b, 810c, 815a, 815b, 820a, 820b, 830, and so on. In some examples, the predicted metrics 2330 include at least one of a metric value associated with temporal workflow, a metric value associated with scheduling, a metric value associated with human resources, a metric value for an adverse event, a metric value for a headcount in a medical environment, or a metric value for traffic in the medical environment. In some examples, the predicted metrics 2330 include at least one of an Efficiency metric value, a Consistency metric value, an Adverse Events metric value, a Case Volume metric value, a First Case Turnovers metric value, a Delay metric value, an OR Traffic metric value, a Room Layout metric value, and a Modality Conversion metric value. In some examples, the predicted metrics 2330 can include the ORA score.

In some examples, with respect to a given medical procedure, predicted metrics 2330 can be predicted for the entire medical procedure, for a period of the medical procedure, for a phase of the medical procedure, for a task of the medical procedure, for a surgeon, for a care team, for a medical staff, and so on. For example, an average predicted metric 2330 can be predicted by the prediction system 2320 for a medical procedure, for a period of the medical procedure, for a phase of the medical procedure, for a task of the medical procedure, for a surgeon, for a care team, for a medical staff, such that the predicted metric 2330 are the average of the dataset based on which the prediction system 2320 is trained excluding the outliers (e.g., greater than 2.5 or more standard deviations of the average). With respect to a given medical procedure, predicted metrics 2330 can be predicted for temporal workflow efficiency, for a number of medical staff members, for time duration of each segment (e.g., phase or task) of the medical procedure, for motion, for room size and layout, for timeline, for non-operative periods or adverse events, and so on. In some examples, the predicted metrics 2330 can be predicted for each time segment (e.g., period, phase, task, and so on) of a medical procedure. Accordingly, for a given medical procedure, predicted metrics 2330 can be predicted for each of two or more multiple time segments (e.g., periods, phases, and tasks) of a medical procedure. In some examples, the predicted metrics 2330 include at least one metric value or a range of metric values for a time interval defined by patient-close to patient wheel out, a time interval defined by turn-over for next case, a time interval defined by patient-in to first-cut for next case, and so on. In some examples, the predicted metrics 2330 can include predicted timestamps (e.g., the start time and the end time) of a time interval corresponding to a period, stage, phase, task, etc. In some examples, the predicted metrics 2330 can include predicted length of a time interval corresponding to a period, stage, phase, task, etc. In some examples, error bounds, uncertainty measures, error margins (e.g., mean test error), and so on can be calculated for the predicted metrics 2330 and provided for display along with the predicted metrics 2330. The error bounds, uncertainty measures, and error margins can provide an indication of the confidence level of the predicted metrics 2330 to allow a user to determine the extent to which the predicted metrics 2330 can be relied.

In some embodiments, predicted metrics 2330 such as the ORA score can be predicted for each procedure, OR, hospital, surgeon, healthcare team, procedure type, over multiple medical procedures. In some embodiments, predicted metrics 2330 can include a metric that is an average of metrics predicted for multiple medical procedures in an OR, in a hospital, by a surgeon, by a healthcare team, for a procedure type, and so on. In some examples, a procedure type of a medical procedure can be defined based at least in part on one or more of a modality (robotic, open, lap, etc.), operation type (e.g., prostatectomy, nephrectomy, etc.), procedure workflow efficiency rating (e.g., high-efficiency, low efficiency, etc.), certain type of hospital setting (e.g., academic, outpatient, training, etc), and so on.

As shown, the prediction system 2320 includes at least one of a predictive model 2322 or a correlation function 2324. The correlation function 2324 includes one or more machine learning models and/or functions that can learn and extract statistically significant and highly correlated aspects or features between historic robotic system data (referred to as second robotic system data) and efficiency-related data obtained in historical medical procedures (referred to as second medical procedures) in medical environments (referred to as second medical environments). The predictive system 2320 includes one or more machine learning models and/or functions (e.g., a prediction model 2322) that can be trained (e.g., updated) to predict metric values 2330 based at least in part on the correlations identified by the correlation function 2324. During deployment, even though the robotic system data 2310 and the metadata 2315 are collected for one or more of a medical procedure, care team, and medical environment that may be different from those for the training data set on which the prediction system 2320 is trained, the prediction system 2320 can apply gained insight from those different medical procedures, care teams, medical environments to the current medical procedure.

As used herein, a medical procedure refers to a surgical procedure or operation performed in a medical environment (e.g., a medical or surgical theater 110a or 110b, OR, etc.) by or using one or more of a medical staff, a robotic system, or an instrument. Examples of the medical staff include surgeons, nurses, support staff, and so on, such as the patient-side surgeon 105a and the assisting members 105b. Examples of the robotic systems include the robotic medical systems or the robot surgical systems described herein. Examples of instruments include the mechanical instrument 110a, the visualization tool 110b, and so on. The theater-wide data collected for a medical procedure also refers or includes theater-wide data collected in a medical environment in which the medical procedure is performed and for one or more of medical staff, robotic system, or instrument performing or used in performing the medical procedure.

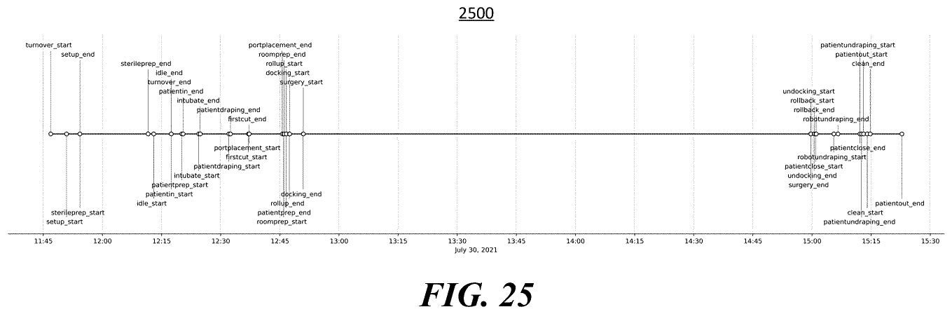

In some examples, the efficiency-related data includes one or more of the theater-wide data (e.g., the sensor output), identified segments (e.g., periods, phases, and tasks), a timeline of the identified segments, metric values calculated for the segments, and so on determined using methods described in FIGS. 1-22. FIG. 25 is an example timeline 2500 including various segments, according to some embodiments. The timeline 2500 is similar to the timeline element 2210. The timeline 2500 includes various segments each indicated using strings to describe the segment and having an associated timestamp (e.g., a start timestamp and an end timestamp). As shown, examples of the segments in the timeline 2500 include turnover, setup, sterilization preparation, intubation, patient in, patient draping, first cut, port placement, docking, surgery start, roll up, room preparation, undocking, rollback, patient undraping, cleaning, and so on. The timeline 2500 and the segments therein can be determined or identified using the theater-wide data as described herein in FIGS. 1-22.

In some examples, the timelines 2400 and 2500 can be determined for each of a plurality of medical procedures in a plurality of medical environments. In some examples, at least some or all of the system events defined or identified in the timeline 2400 are different from the segments defined or identified in the timeline 2500. In some examples, a system event defined or identified in the timeline 2400 is the same as a segment defined or identified in the timeline 2500. Correlations between the aspects and features of the timelines 2400 and 2500 can be extracted.

In some examples, the historic robotic system data and efficiency-related data obtained in the same medical procedures and the timestamps thereof can be time synchronized and integrated before the correlations are extracted using the correlation function 2324. The time synchronizing of the historic robotic system data and efficiency-related data is a pre-training operation performed before training the predictive model 2322.

In some examples, a system clock of a theater-wider sensor and a system clock of the robotic system are synchronized to a reference clock, such as the clock of the theater-wider sensor, the robotic system, the prediction system 2320, the electronics/control console 145, the systems 190a, 190b, 450b, or another suitable third party clock, such that the timestamps of the theater-wider sensor and the robotic system are already synchronized as the theater-wide data and the robotic system data 2310 are collected.

In some examples, both the theater-wider data and the robotic system data 2310 are received and timestamped by a same server (e.g., the prediction system 2320, the electronics/control console 145, the systems 190a, 190b, 450b, or the like) in real time, to ensure that the timestamps of the theater-wider sensor and the robotic system data 2310 are aligned. The server can timestamp both the theater-wider data and the robotic system data 2310 according to the system clock of the server as the theater-wider data and the robotic system data 2310 are being received or collected.

In some examples, time-synchronization can include aligning timestamps of the theater-wider data and the robotic system data 2310 according to detection of a common event, which is an event that is shared by the theater-wider data and the robotic system data 2310. For example, a same event such as a “rolling up” event (e.g., pushing a robotic system forward) can be defined in both the timelines 2400 and 2500. The robotic system data 2310 can include the “rolling up” event with a timestamp in the timeline 2400 that defines each of a start time or an end time, and the efficiency-related data can also include a segment (e.g., a phase) defined as “rolling up” with a timestamp in the timeline 2500 that defines each of a start time or an end time. In the examples in which both the timelines 2400 and 2500 include the same “rolling up” event, the timestamps that define the start time for the “rolling up” event in both the timelines 2400 and 2500 can be aligned, and the timestamps that define the end time for the “rolling up” event in both the timelines 2400 and 2500 can be aligned. In some examples, timestamps defining at least one of a start time or end time of two or more such common events can be aligned. In some examples, timestamps defining at least one of a start time or end time of two or more events can be aligned. The rest of the timelines 2400 and 2500 can be accordingly synchronized.

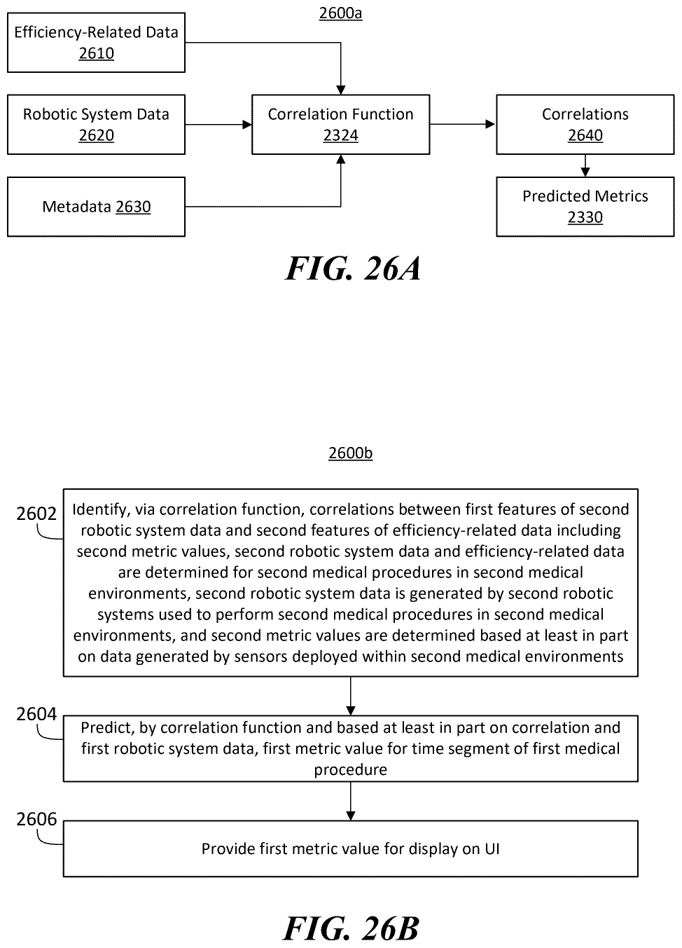

FIG. 26A is a block diagram of an example method 2600a for determining correlations 2640 between efficiency-related data 2610 and robotic system data 2310 and using the correlations 2640 to determine predicted metrics 2330, according to some embodiments. FIG. 26B is a flowchart diagram of an example method 2600b for determining correlations 2640 between efficiency-related data 2610 and robotic system data 2310 and using the correlations 2640 to determine predicted metrics 2330, according to some embodiments. In some implementations, historic efficiency-related data 2610, historic robotic system data 2620, and metadata 2630 associated therewith are applied to the correlation function 2324 to generate output correlations 2640. The correlations 2640 can be applied to robotic system data 2310 (e.g., first robotic system data) of a current medical procedure to identify one or more predicted metrics 2330 (e.g., a first metric value) highly correlated to the robotic system data 2310, and provide such predicted metrics 2330 for display on a UI. The robotic system data 2620 is similar to the robotic system data 2310, except that the robotic system data 2620 is generated for historical medical procedures. The historic efficiency-related data 2610, historic robotic system data 2620, and metadata 2630 can be stored in one or more suitable storage devices or databases and can be retrieved by the correlation function 2324 to learn the correlations 2640.

The metadata 2315 and 2630 includes information of various aspects and attributes of medical procedures, including at least one of identifying information of the medical procedure, identifying information of one or more medical environments (e.g., theaters, ORs, hospitals, and so on) in which the medical procedure is performed, identifying information of medical staff by whom the medical procedure is performed, the experience level of the medical staff, schedules of the medical staff and the medical environments, patient complexity of patients subject to the medical procedure, patient health parameters or indicators, identifying information of one or more robotic systems or instruments used in the medical procedure, identifying information of one or more sensors used to capture the theater-wide data or the multimodal data, and so on.

In some examples, the identifying information of a medical procedure includes at least one of a name or type of each of the medical procedure, a time at which or a time duration in which each of the medical procedure is performed, or a modality of each of the medical procedure. In some examples, the identifying information of the one or more ORs includes a name of each of the one or more ORs. In some examples, the identifying information of the one or more hospitals includes a name of each of the one or more hospitals. In some examples, the identifying information of the medical staff members includes a name, specialty, job title, ID and so on of each of one or more surgeons, nurses, healthcare team name, and so on. In some examples, the experience level of the medical staff members includes a role, length of time for practicing medicine, length of time for performing certain types of medical procedures, length of time for using a certain type of robotic systems, certifications, and credentials of each of one or more surgeons, nurses, healthcare team name or ID, and so on. The schedules of the medical staff and the medical environments include allocation of the medical staff and the medical environments to perform certain procedures (e.g., defined by types of surgery, surgery name, surgery ID, or surgery reference number, specialty, modality), names of medical staff members, and corresponding time.

In some examples, patient complexity refers to conditions that a patient has that may influence the care of other conditions. In some examples, patient health parameters or indicators include various parameters or indicators such as body mass index (BMI), percentage body fat (% BF), blood serum cholesterol (BSC), and systolic (SBP), height, stage of sickness, organ information, outcome of the medical procedure, and so on. In some examples, the identifying information of the one or more robotic systems or instruments includes at least one of a name, model, or version of each of the one or more robotic systems or instruments or an attribute of each of the one or more robotic systems or instruments. In some examples, the identifying information of at least one sensor includes at least one of a name of each of the at least one sensor or a modality of each of the at least one sensor.

In some examples, the metadata 2315 and 2630 can be stored in a memory device of a suitable computing system or a database. The memory device or the database can be provided for a scheduling or work allocation application that schedules the medical staff and the medical procedures in medical environments. For example, a user can input using an input system the metadata 2315 and 2630, or the metadata 2315 and 2630 can be automatically generated using an automated scheduling application. The types of the theater-wide data captured for the same procedure time or scheduled time, in the same medical environment, with the same procedure name, with the same robot or instrument, by the same medical staff, or so on can be associated and stored with the corresponding metadata 2315 and 2630.

At 2602, the correlation function 2324 identifies the correlations 2640 between first features of robotic system data 2620 and second features of the efficiency-related data 2610. The efficiency-related data 2610 includes one or more second metric values. In some examples, a first feature includes low-level information or features such as tokens, tensors, embeddings, and so on extracted from the robotic system data 2620. In some examples, a first feature includes high-level features such as the identified system events (e.g., docking event, operator head-in or head-out event, an instrument attachment or removal event, an instrument change event, a draping-start event or a sterile adapter attachment event, and so on), a timestamp of a system event, compound system event or the time interval between timestamps of two system events (e.g., between a head-in event and a head-out event, between an instrument attachment event and an instrument removal event, between a draping start event and a draping end event, and so on), the order of system events, relative temporal placement of one system event to one or more other system events, and so on.

Training and test dataset including the historic efficiency-related data 2610, historic robotic system data 2620 (e.g., the second robotic system data), and metadata 2630 are collected for historical medical procedures (e.g., the second medical procedures) performed in the second medical environments. For example, the robotic system data 2620 is generated by second robotic systems used to perform the second medical procedures in the second medical environments. The efficiency-related data 2610 (e.g., including the one or more second metric values) includes or is determined based at least in part on medical environment data, such as the multimodal data (e.g., the depth data or the 3D data) generated by theater-wide sensors (e.g., the depth acquiring sensors) deployed within the second medical environments.

The historic efficiency-related data includes one or more of the theater-wide data, identified segments (e.g., periods, phases, and tasks), a timeline of the identified segments, metric values calculated in the manner described herein for the segments, and so on. In other words, the historic efficiency-related data includes the theater-wide data (e.g., depth data, visual data, etc.) and the information determined using the theater-wide data. The output correlations can identify aspects of the historic robotic system data that highly correlate to the historic efficiency-related data (e.g., the metric values).

In some examples, a second feature includes low-level information features such as tokens, tensors, embeddings, and so on extracted from the efficiency-related data 2610, which includes the theater-wide data, identified segments, timeline of identified segments, metric values calculated for the segments, and so on. In some examples, a second feature includes high-level features such as a type of a segment (e.g., nonoperative periods 310a, 310b, 310c, 310d, tasks 320a, 320b, 320c, 320d, 320f, and 320e, phases 520, 525, 510, and 515), a length of a segment, the timestamps of the beginning and end of a segment, the order of segments, relative temporal placement of one segment to one or more other segments, metric values or ranges of metric values determined for each segment or for the entire medical procedure, and so on.

In some examples, the correlations 2640 between the first features of the robotic system data 2620 and the second features of the efficiency-related data 2610 is identified based at least in part on or conditioned upon the metadata 2630 of the medical procedures for which the robotic system data 2620 and the efficiency-related data 2610 are collected. In some examples, the metadata 2630 is provided to the correlation function 2324. The metadata 2630 can generally be in text or string character format to describe the various aspects of the medical procedures in relation to the robotic system data 3104 or the instrument data 3106. Such aspects may include identifying information of the historic medical procedures, identifying information of the medical environments in which the historic second medical procedures are performed, identifying information of medical staff by which the medical procedures are performed, experience level of the medical staff, patient complexity, patient health parameters or indicators, or identifying information of the robotic medical systems or instruments used in the historic medical procedures, and so on. In other words, the metadata 2630 can provide additional context that may be useful for extracting the correlations 2640, in the case in which the relationship between the robotic system data 3104 and the metadata 3108 can be more clearly defined, indirectly, via the metadata 2630.

In some examples, the correlation function 2324 can extract the correlations 2640 between the first features (e.g., any type of the low-level features and high-level features) of the robotic system data 2620 to a metric value included in the efficiency-related data 2610, for an identified segment (e.g., for a period, phase, or task). In other words, the correlations 2640 include the correlations between the first features of the robotic system data 2620 and metric values for each of a plurality of segments identified of the historic medical procedures. In some examples, the correlations 2640 include correlation score indicating a degree of correlation between a first feature and a second feature. First and second features exhibiting greater degree of correlation are assigned a high correlation score by the correlation function 2324, and first and second features exhibiting lesser degree of correlation are assigned a low correlation score by the correlation function 2324.

At 2604, during deployment in which current robotic system data 2310 (e.g., first robotic system data) is collected, the correlation function 2324 predicts or estimates, based at least in part on the correlations 2640 and the robotic system data 2310, the predicted metrics 2330 (e.g., the first metric value) for a segment of the current medical procedure (the first medical procedure). In some examples, the correlation function 2324 predicts or estimates, based at least in part on the correlations 2640, the robotic system data 2310, and metadata 2315 for the current medical procedure, the predicted metrics 2330 (e.g., the first metric value) for a segment of the current medical procedure (the first medical procedure). Based at least in part on the correlations 2640, the correlation function 2324 can predict the predicted metrics 2330 for a particular segment (e.g., period, phase, and task) using the robotic system data 2310 (and in some cases the metadata 2315) of the current medical procedure without relying on any efficiency-related data for the current medical procedure, to provide insight into the current medical procedure. At 2606, the predicted metrics 2330 can be displayed using a suitable UI.

The correlation function 2324 can be used as the objective function to train the prediction model 2322. For example, the correlation function 2324 can include any suitable correlation methods or functions such as a convolution correlation function, a cross-correlation function, an autocorrelation function. In some examples, the correlation function 2324 can be implemented using one or more machine learning models such as neural networks (e.g., deep neural networks (DNNs), convolutional neural networks (CNNs), etc.).

The machine learning models of the correlation function 2324 can be trained (e.g., updated) using the efficiency-related data 2610 and robotic system data 2620. For example, for a given historical medical procedure (e.g., a second medical procedure), the correlation function 2324 applies the previously determined correlations 2640 to the robotic system data 2620 of the medical procedure, which outputs a predicted metric similar to described above relative to 2604. The predicted metric is compared with the actual metric of the efficiency-related data 2610 determined for the same medical procedure. An objective function (e.g., a gain function, a cost function, a loss function, and so on) can be deployed to determine the difference between the predicted metric and the ground truth, where the difference can be used to train (e.g., update) the machine learning models of the predictive model 2322 as described below, and the correlation function 2324 can be a choice for the objective function to optimize to learn the parameters of the predictive model 2322.

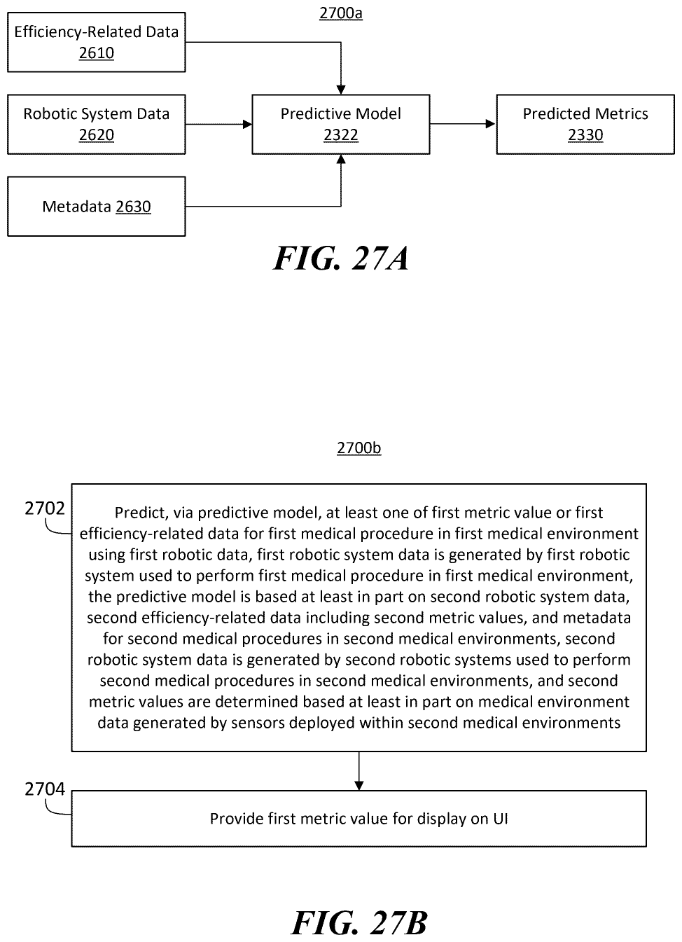

FIG. 27A is a block diagram of an example method 2700a for predicting predicted metrics 2330, according to some embodiments. FIG. 27B is a flowchart diagram of an example method 2700b for predicting predicted metrics 2330, according to some embodiments. In some implementations, historic efficiency-related data 2610, historic robotic system data 2620, and metadata 2630 associated therewith can be used to train (e.g., update) the predictive model 2322 to predict or estimate the predicted metrics 2330. For example, given a dataset of the historic efficiency-related data 2610, a portion (e.g., 90%) of the dataset can be used to train the prediction model 2322, and the remaining portion (e.g., 10%) of the dataset can be used for tests and validation for the prediction model 2322. The prediction model 2322, upon being trained, can apply the robotic system data 2310 and the metadata 2315 and predict at least one of predicted efficiency-related data (e.g., a timeline of the identified segments) or an overall metric corresponding to the entirety of the robotic system data 2310 (e.g., the entirety of the first medical procedure).

At 2702, during deployment in which current robotic system data 2310 (e.g., first robotic system data) is collected, the predictive model 2322 predicts or determines the predicted metrics 2330 (e.g., first metric value) and in some cases a predicted efficiency-related data (e.g., a first efficiency-related data) for a current medical procedure (e.g., first medical procedure) in a first medical environment using current robotic system data 2310 (e.g., first robotic system data). The current robotic system data 2310 is generated by a first robotic system used to perform the current medical procedure in the first medical environment. In some examples, the predicted efficiency-related data refers to a type or name of a segment, a length of a segment, or a timeline composed of a plurality of segments, that is not determined using any actual sensors, but is instead predicted using the robotic system data 2310. The predicted metrics 2330 includes a metric (e.g., an ORA score 830) that reflects an overall integrated metric for the medical procedure or multiple medical procedures. At 2704, the predicted metrics 2330 can be displayed using a suitable UI.

The predictive model 2322 is based at least in part on (e.g., trained, generated, or updated based at least in part on) historic robotic system data 2620 (e.g., the second robotic system data), historic efficiency-related data 2610 (e.g., second efficiency-related data including one or more second metric values), and metadata 2630 for the historic medical procedures performed in second medical environments. For example, the robotic system data 2620 is generated by second robotic systems used to perform the historic medical procedures in the second medical environments. The efficiency-related data 2610 (e.g., the one or more second metric values) includes or is determined based at least in part on medical environment data such as the multimodal data (e.g., the depth data or the 3D data) generated by theater-wide sensors (e.g., the depth acquiring sensors) deployed within the second medical environments.

The historic efficiency-related data 2610 includes one or more of the theater-wide data, identified segments (e.g., periods, phases, and tasks), a timeline of the identified segments, metric values calculated in the manner described herein for the segments, and so on. In other words, the historic efficiency-related data includes the theater-wide data (e.g., depth data, visual data, etc.) and the information determined using the theater-wide data.

The predictive model 2322 can be implemented using any suitable mathematical model, statistical model, deep learning algorithms, machine learning models, or a combination thereof that is used, trained, or updated to determine a predicted metric based at least in part on robotic system data. For example, the predictive model 2322 can include any suitable functions or models (e.g., linear models, representative models, and so on) that can infer or predict an outcome based at least in part on robotic system data. In some examples, the predictive model 2322 can be implemented using one or more machine learning models such as neural networks (e.g., DNNs, CNNs, etc.).

The machine learning models of the predictive model 2322 can be trained (e.g., updated) using the efficiency-related data 2810, the robotic system data 2620, and the metadata 2630. For example, for a given historical medical procedure (e.g., a second medical procedure), the predictive model 2322 predicts an overall predicted metric (e.g., an ORA score 830) similar to described above relative to 2702. The predicted metric is compared with the actual metric (e.g., the actual ORA score 830) of the efficiency-related data 2610 determined for the same medical procedure or multiple medical procedures. An objective function (e.g., a gain function, a cost function, a loss function, and so on) can be deployed to determine the difference between the predicted metric and the ground truth, where the difference can be used to train (e.g., update) the machine learning models of the predictive model 2322. For example, a loss function or a cost function can be used to determine the loss or cost (e.g., a square loss, a MSE, and so on) between the predicted metric and the ground truth of the actual metric of the efficiency-related data 2610. One or more weights, biases, parameters, or configurations of the machine learning models of the predictive model 2322 can updated to minimize the loss. For example, a gain function can be used to determine the gain between the predicted metric and the ground truth of the actual metric of the efficiency-related data 2610. One or more weights, biases, parameters, or configurations of the machine learning models of the predictive model 2322 can updated to maximize the gain. In some examples, the predictive model 2322 includes a linear head, such that the output of the neural network is provided to the linear head, which outputs or the predicted metrics 2330. The linear head can be defined by:

$$y = {a}^{T} x + b , \qquad ( 3 )$$

where y is the desired prediction given a set of predictors in x, b is the intercept. The vector a contains the coefficient corresponding to each predictor.

$$a = [ \begin{matrix}last follow \\ first follow\end{matrix} \vert … ] [ \begin{matrix}\begin{matrix}0.05 \\ 0.095\end{matrix} \\ …\end{matrix} ] . \qquad ( 4 )$$

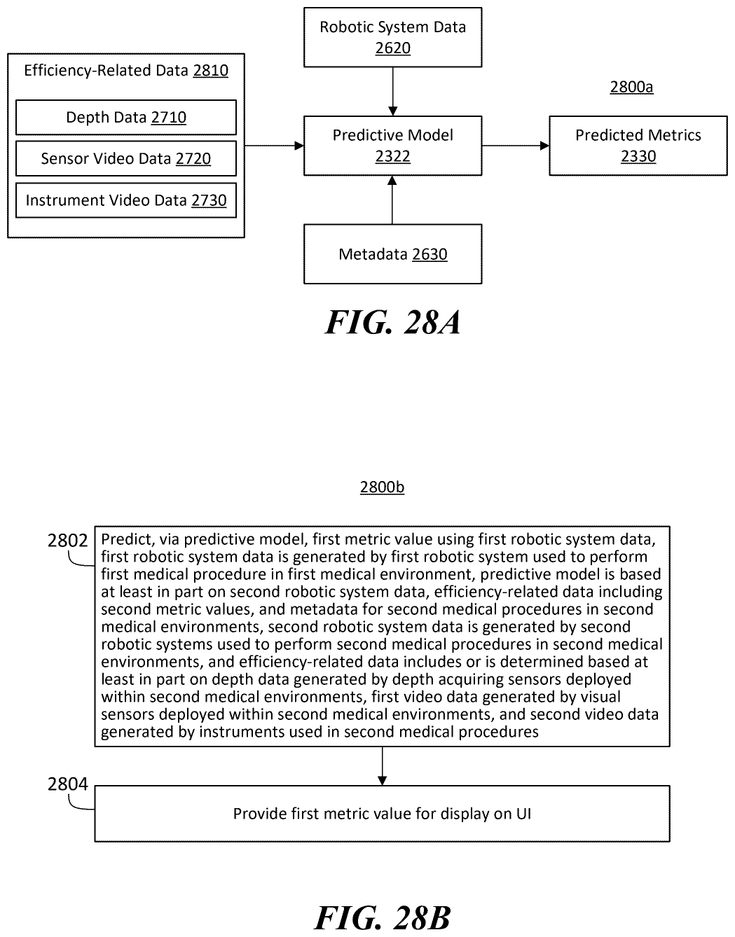

Given may data points stacked on top of each other, we arrive in matrix form of the linear model Y=Xa. FIG. 28A is a block diagram of an example method 2800a for predicting predicted metrics 2330, according to some embodiments. FIG. 28B is a flowchart diagram of an example method 2700b for predicting metrics 2330 (e.g., predicted metrics 2330), according to some embodiments. In some implementations, historic efficiency-related data 2810, historic robotic system data 2620, and metadata 2630 associated therewith can be used to train (e.g., update) the predictive model 2322 to predict or estimate the predicted metrics 2330. For example, given a dataset of the historic efficiency-related data 2810, a portion (e.g., 90%) of the dataset can be used to train the prediction model 2322, and the remaining portion (e.g., 10%) of the dataset can be used for tests and validation for the prediction model 2322. The prediction model 2322, upon being trained, can apply the robotic system data 2310 and the metadata 2315 and predict an overall metric corresponding to the entirety of the robotic system data 2310 (e.g., the entirety of the first medical procedure).

In some examples, the historic efficiency-related data 2810 includes one or more of the theater-wide data (e.g., the depth data 2710), identified segments (e.g., periods, phases, and tasks), a timeline of the identified segments, metric values calculated in the manner described herein for the segments, as well as sensor video data 2720 (e.g., first video data) and instrument video data 2730 (e.g., second video data), and so on. The depth data 2710 is collected by depth acquiring sensors deployed within a medical environment and can include depth videos or frames over time. The sensor video data 2720 is collected by visual image sensors deployed within a medical environment. As described herein, each of the sensors 170a and 170c can be a visual image sensor (e.g., color or grayscale image sensors), depth-acquiring sensors (e.g., via stereoscopically acquired visual image pairs, via time-of-flight with a laser rangefinder, structural light, etc.), or a multimodal sensor including a combination of a visual image sensor and a depth-acquiring sensor (e.g., a red-green-blue depth RGB-D sensor). Thus, sensors such as the sensors 170a and 170c can output the depth data 2710 and the sensor video data 2720.

In some examples, the instrument video data 2730 can be collected by instruments used in a medical procedures. In some examples, an instrument (e.g., the visualization tool 110b) can be manually operated by a medical personnel (e.g., the surgeon 105a). In some examples, an instrument (e.g., the visualization tools 140a, 140b, 140c, and 140d) can be attached to a robotic system (e.g., a robotic surgical system) such as on the arms 135a, 135b, 135c, and 135d thereof. Examples of the instrument can include a laparoscopic ultrasound, visual image acquiring endoscope, and so on. The instrument video data 2730 can include any 2D representation in video format.

Accordingly, the predictive model 2322 can be trained using additional video data sources, which can provide additional context beyond the information that can be exposed through the robotic system data 2620, the depth data 2710, and the metadata 2630. In the implementations in which additional video data 2720 and 2730 are available, such additional video data 2720 and 2730 can be leveraged to further improve the accuracy and efficiency for training the predictive model 2322.

At 2802, during deployment in which current robotic system data 2310 (e.g., first robotic system data) is collected, the predictive model 2322 predicts the predicted metrics 2330 (e.g., first metric value) for a current medical procedure (e.g., first medical procedure) in a first medical environment using current robotic system data 2310 (e.g., first robotic system data). The predicted metrics 2330 includes a metric (e.g., an ORA score 830) that reflects an overall integrated metric for the current medical procedure (or multiple medical procedures). The current robotic system data 2310 is generated by a first robotic system used to perform the current medical procedure in the first medical environment. In some examples, during deployment, one or more of current sensor video data (e.g., similar to the sensor video data 2720) or instrument video data (e.g., similar to the instrument video data 2730) of the current medical procedure can be received by the predictive model 2322, if available, to further improve accuracy and efficiency of generating the predicted metrics 2330. In some examples, the output of the predictive model 2322 during deployment can further include predicted efficiency-related data, which can include a type or name of a segment, a length of a segment, or a timeline composed of a plurality of segments, that is not determined using any actual sensors, but is instead predicted using the robotic system data 2310. At 2804, the predicted metrics 2330 can be displayed using a suitable UI.

The predictive model 2322 is based at least in part on (e.g., trained, generated, or updated based at least in part on) historic robotic system data 2620 (e.g., the second robotic system data), historic efficiency-related data 2810 (e.g., second efficiency-related data including one or more second metric values), and metadata 2630 for the historic medical procedures performed in second medical environments. For example, the robotic system data 2620 is generated by second robotic systems used to perform the historic medical procedures in the second medical environments. The historic efficiency-related data includes one or more of the theater-wide data (e.g., the depth data 2710), identified segments (e.g., periods, phases, and tasks), a timeline of the identified segments, metric values calculated in the manner described herein for the segments, the sensor video data 2720, the instrument video data 2730, and so on.

As described herein, the predictive model 2322 can be implemented using any suitable mathematical model, statistical model, deep learning algorithms, machine learning models, or a combination thereof that is used, trained, or updated to determine a predicted metric based at least in part on robotic system data. For example, the predictive model 2322 can include any suitable functions or models (e.g., linear models, representative models, and so on) that can infer or predict an outcome based at least in part on robotic system data. In some examples, the predictive model 2322 can be implemented using one or more machine learning models such as neural networks (e.g., DNNs, CNNs, etc.).

The machine learning models of the predictive model 2322 can be trained (e.g., updated) using the efficiency-related data 2810, the robotic system data 2620, and the metadata 2630. For example, for a given historical medical procedure (e.g., a second medical procedure), the predictive model 2322 predicts an overall predicted metric (e.g., an ORA score 830) similar to described above relative to 2802. The predicted metric is compared with the actual metric (e.g., the actual ORA score 830) of the efficiency-related data 2810 determined for the same medical procedure. As described, an objective function (e.g., a gain function, a cost function, a loss function, and so on) can be deployed to determine the difference between the predicted metric and the ground truth, where the difference can be used to train (e.g., update) the machine learning models of the predictive model 2322, efficiency-related data In some examples, the predictive model 2322 includes a linear head, such that the output of the neural network is provided to the linear head, which outputs or the predicted metrics 2330. The linear head can be defined by expression (3) and (4).

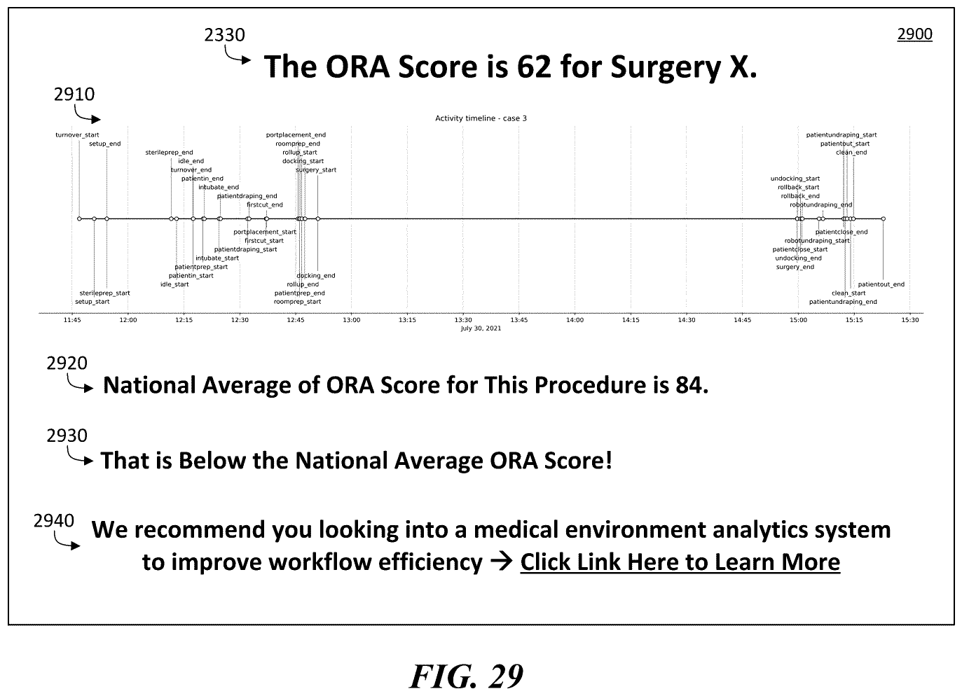

FIG. 29 illustrates an example UI 2900 configured to display the predicted metric 2330 and other associated information, according to various embodiments. As shown, the UI 2900 displays the predicted metric 2330 determined in the manner described herein, including those described with reference FIGS. 23-28B. The computing system implementing the prediction system 2320 can provide (e.g., send, communicate, or transfer via a suitable network) the information to be displayed to another computing system having an output device or display configured to display such information in the UI 2900. Examples of the output device or display include one or more of the display 125, 150, and 160a, a display that outputs information for the applications 450f, and display communicably coupled to the processing systems 190a, 190b, and 450b, display device or touch screen device of the input/output devices 3020, and so on. In other words, the UI 2900 can be displayed using displays 125, 150, 160a, etc. that can be located within the surgical theaters 100a and 100b for real-time feedback to the medical personnel during the medical procedure. The UI 2900 can be displayed using displays for the applications 450f that can be located remote from the surgical theaters 100a and 100b to provide discovery of information to consultants and students for studying and analyzing the information of a medical procedure at any time after or concurrent with the medical procedure and to remote support staff providing real-time assistance to the medical procedure.

A name of the medical procedure (e.g., “Surgery X”) can be displayed in the UI 2900. In some examples, the UI 2900 can display a timeline 2910 of segments of the current (first) medical procedure. The timeline 2910 can be a predicted timeline of the predicted efficiency-related data as described in FIG. 27A. The displayed predicted metric 2330 can also include a metric value for one or more segments of the current medical procedure.

In some examples, for comparison, historical data (e.g., statistics) for the at least one segment or the entire medical procedure for which the predicted metric 2339 is determined can be displayed in the UI 2900. In the UI 2900, a national average ORA score 2920 for the same procedure is displayed, although in other UIs, averages, standard deviations, thresholds, and other statistics for the same procedure or any segment thereof relative to different procedures, different types of robotic systems, different care teams, different ORs, different hospitals, different regions, different countries, at different times, and so on can be displayed.

In some examples, an alert or notification 2930 can be displayed by the UI 2900 in response to determining that the predicted metric 2330 for a segment or for the entire medical procedure crosses (e.g., is greater than or is less than) a predetermined threshold for that segment or medical procedure. For example, the notification 2930 indicates that the ORA score 62 for the current medical procedure is below the threshold of the national average ORA score. Such notification 2930 can indicate predicted efficiency or inefficiency for that segment or medical procedure. In some examples, a recommendation 2940 corresponding to the predicted metric 2330 and generated by the prediction system 2320 can be displayed in the UI 2900. In some examples, a predicted metric value or a range of predicted metric values can be mapped to one or more predetermined recommendations. For example, a predicted metric value indicating low OR efficiency is mapped to a recommendation for deploying medical environment analytics system, e.g., as in the recommendation 2940, for further analysis. The recommendation can include a hyperlink to a network page providing information of the medical environment analytics system.

In some examples, two or more of the predicted metric 2330, the at least one segment or medical procedure for which the predicted metric value is determined, the timeline 2910, the historical and statistical data 2920, the notification 2930, or the recommendation 2940 can be displayed in the same UI 2900. In some examples, two or more of the predicted metric 2330, the at least one segment or medical procedure for which the predicted metric value is determined, the timeline 2910, the historical and statistical data 2920, the notification 2930, or the recommendation 2940 can be displayed in two different UI.

### Computer System

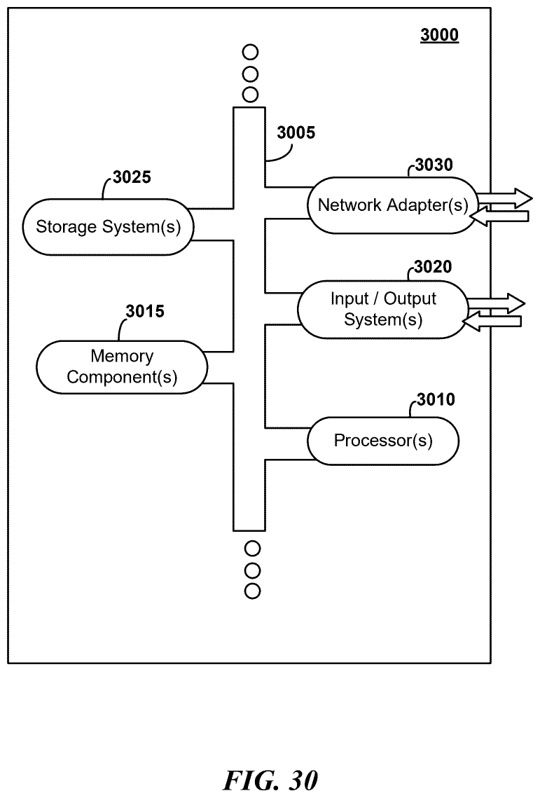

FIG. 30 is a block diagram of an example computer system 3000 as may be used in conjunction with some of the embodiments. In some examples, each of the processing systems 450b can be implemented using the computing system 3000. In some examples, the application 450f can be executed using the computing system 3000. The computing system 3000 may include an interconnect 3005, connecting several components, such as, e.g., one or more processors 3010, one or more memory components 3015, one or more input/output systems 3020, one or more storage systems 3025, one or more network adaptors 3030, etc. The interconnect 3005 may be, e.g., one or more bridges, traces, busses (e.g., an ISA, SCSI, PCI, I2C, Firewire bus, etc.), wires, adapters, or controllers.

The one or more processors 3010 may include, e.g., a general-purpose processor (e.g., x86 processor, RISC processor, etc.), a math coprocessor, a graphics processor, etc. The one or more memory components 3015 may include, e.g., a volatile memory (RAM, SRAM, DRAM, etc.), a non-volatile memory (EPROM, ROM, Flash memory, etc.), or similar devices. The one or more input/output devices 3020 may include, e.g., display devices, keyboards, pointing devices, touchscreen devices, etc. The one or more storage devices 3025 may include, e.g., cloud-based storages, removable Universal Serial Bus (USB) storage, disk drives, etc. In some systems memory components 3015 and storage devices 3025 may be the same components. Network adapters 3030 may include, e.g., wired network interfaces, wireless interfaces, Bluetooth™ adapters, line-of-sight interfaces, etc.

One will recognize that only some of the components, alternative components, or additional components than those depicted in FIG. 30 may be present in some embodiments. Similarly, the components may be combined or serve dual-purposes in some systems. The components may be implemented using special-purpose hardwired circuitry such as, for example, one or more ASICs, PLDs, FPGAs, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 3030. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 3015 and one or more storage devices 3025 may be computer-readable storage media. In some embodiments, the one or more memory components 3015 or one or more storage devices 3025 may store instructions, which may perform or cause to be performed various of the operations discussed herein. In some embodiments, the instructions stored in memory 3015 can be implemented as software and/or firmware. These instructions may be used to perform operations on the one or more processors 3010 to carry out processes described herein. In some embodiments, such instructions may be provided to the one or more processors 3010 by downloading the instructions from another system, e.g., via network adapter 3030.

For clarity, one will appreciate that while a computer system may be a single machine, residing at a single location, having one or more of the components of FIG. 30, this need not be the case. For example, distributed network computer systems may include multiple individual processing workstations, each workstation having some, or all, of the components depicted in FIG. 30. Processing and various operations described herein may accordingly be spread across the one or more workstations of such a computer system. For example, one will appreciate that a process amenable to being run in a single thread upon a single workstation may instead be separated into an arbitrary number of sub-threads across one or more workstations, such sub-threads then run in serial or in parallel to achieve a same, or substantially similar, result as the process run within the single thread. Similarly, one will appreciate that while a non-transitory computer readable medium may stand alone (e.g., in a single USB storage device), or reside within a single workstation (e.g., in the workstation's random access memory or disk storage), such a medium need not reside at a single geographic location, but may include, e.g., multiple memory storage units residing across geographically separated workstations of a computer system in network communication with one another or across geographically separated storage devices.

### Remarks

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

What is claimed is:

1. A prediction system, comprising:
one or more processors, coupled with memory, configured to:
determine, via at least one machine learning model, a first metric value using first robotic system data, wherein the first robotic system data is generated by a first robotic system used to perform a first medical procedure in a first medical environment, wherein the at least one machine learning model is based at least in part on second robotic system data and efficiency-related data comprising one or more second metric values for a plurality of second medical procedures in a plurality of second medical environments, wherein the second robotic system data is generated by a plurality of second robotic systems used to perform the plurality of second medical procedures in the plurality of second medical environments, and wherein the one or more second metric values is determined based at least in part on the second robotic system data and medical environment data generated by sensors deployed within the plurality of second medical environments; and
provide the first metric value for display on a User Interface (UI).

2. The prediction system of claim 1, wherein
the one or more processors to time synchronize the second robotic system data and the efficiency-related data for each of the plurality of second medical procedures;
the second robotic system data and the efficiency-related data for each of the plurality of second medical procedures is received by a same server in real time; and
the server timestamps the second robotic system data and the efficiency-related data according to a clock of the server;
time synchronizing the second robotic system data and the efficiency-related data comprises:
identifying a common event between the second robotic system data and the efficiency-related data; and
aligning at least one first timestamp of the second robotic system data defining the common event with at least one second timestamp of the efficiency-related data defining the common event.

3. The prediction system of claim 1, wherein
the at least one machine learning model comprises a correlation function; and
the one or more processors is configured to identify, via the correlation function, correlations between first features of second robotic system data and second features of efficiency-related data.

4. The prediction system of claim 3, wherein
the first features of the second robotic system data comprises at least one of tokens, tensors, or embeddings extracted from the second robotic system data; and
the second features of the efficiency-related data comprises at least one of tokens, tensors, or embeddings extracted from the efficiency-related data.

5. The prediction system of claim 3, wherein the correlations between the first features of second robotic system data and the second features of efficiency-related data is identified based at least in part on or conditioned upon metadata of the plurality of second medical procedures.

6. The prediction system of claim 3, wherein
identifying, via the correlation function, the correlations between the first features of the second robotic system data and the second features of the efficiency-related data comprises identifying, via the correlation function, the correlations between the first features of the second robotic system data and the one or more second metric values for the second medical procedures.

7. The prediction system of claim 3, wherein the one or more second metric values are for each of a plurality of segments of each of the second medical procedures determined based at least in part of the medical environment data generated by the sensors.

8. The prediction system of claim 1, wherein
the at least one machine learning model comprises a predictive model;
the first metric value is predicted for an entirety of the first medical procedure;
the predictive model is configured to determine first efficiency-related data using the first robotic system data, the first efficiency-related data comprises a predicted type or name of a segment of the first medical procedure, a length of the segment, or a timeline of a plurality of segments of the first medical procedure;

9. The prediction system of claim 8, wherein the efficiency-related data comprises or is determined based at least in part on:
depth data generated by depth acquiring sensors deployed within the plurality of second medical environments;
first video data generated by visual sensors deployed within the plurality of second medical environments; and
second video data generated by instruments used in the plurality of second medical procedures.

10. The prediction system of claim 9, wherein the first video data comprises video data collected by one or more of:
visual image sensors arranged within the second medical environments; or
egocentric sensors worn by medical personnel performing the plurality of second medical procedures.

11. The prediction system of claim 9, wherein the instruments comprises at least one of:
instruments used manually by medical personnel performing the plurality of second medical procedures;
instruments provided on the plurality of second robotic systems;
laparoscopic ultrasound devices; or
visual image acquiring endoscopes.

12. The prediction system of claim 1, wherein the UI displays:
the first metric value; and
at least one of:
a timeline of segments of the first medical procedure;
names of the segments of the first medical procedure;
historical data and statistics for the first metric value;
alert and notification comprising or determined base at least in part of the first metric value;
a recommendation mapped to the first metric value.

13. A prediction system configured to determine a first metric value using first robotic system data, wherein the first robotic system is generated by a first robotic system used to perform a first medical procedure in a first medical environment, the prediction system comprising:
one or more processors running at least one machine learning model, the one or more processors coupled with memory, to:
receive second robotic system data and efficiency-related data comprising one or more second metric values for a plurality of second medical procedures in a plurality of second medical environments, wherein the second robotic system data is generated by a plurality of second robotic systems used to perform the plurality of second medical procedures in the plurality of second medical environments, and wherein the one or more second metric values is determined based at least in part on the second robotic system data and medical environment data generated by sensors deployed within the plurality of second medical environments; and
train the at least one machine learning model based at least in part on the second robotic system data and the efficiency-related data for the plurality of second medical procedures in the plurality of second medical environments.

14. The prediction system of claim 13, wherein the one or more processors to time synchronize the second robotic system data and the efficiency-related data for each of the plurality of second medical procedures.

15. The prediction system of claim 13, wherein
the prediction system comprises a correlation function comprising the one or more machine learning models;
the correlation function is trained using the second robotic system data and the efficiency-related data of the plurality of second medical procedures;
training the correlation function comprises:
predicting, by the correlation function and based at least in part on correlations and the second robotic system data for one of the plurality of second medical procedures, a predicted metric value for a time segment of the one of the plurality of second medical procedures;
determining a loss between the predicted metric value for the time segment and an actual metric value of the efficiency-related data of the one of the plurality of second medical procedures; and
updating the correlation function based at least in part on the loss.

16. The prediction system of claim 13, wherein
the prediction system comprises a predictive model comprising the one or more machine learning models;
the predictive model is trained using the second robotic system data and the second efficiency-related data of the plurality of second medical procedures;
predicting, by the predictive model and based at least in part on the second robotic system data and the efficiency-related data for one of the plurality of second medical procedures, a predicted metric value for the one of the plurality of second medical procedures;
determining a loss between the predicted metric value and an actual metric value of the second efficiency-related data of the one of the plurality of second medical procedures; and
updating the predictive model based at least in part on the loss.

17. The prediction system of claim 13, wherein
the prediction system comprises a predictive model comprising the one or more machine learning models;
the predictive model is trained using the second robotic system data, the data generated by the sensors deployed within the plurality of second medical environments, first video data generated by visual sensors deployed within the plurality of second medical environments, and second video data generated by instruments used in the plurality of second medical procedures
predicting, by the predictive model and based at least in part on the second robotic system data and the efficiency-related data for one of the plurality of second medical procedures, a predicted metric value for the one of the plurality of second medical procedures;
determining a loss between the predicted metric value and an actual metric value of the second efficiency-related data of the one of the plurality of second medical procedures; and
updating the predictive model based at least in part on the loss.

18. A prediction system configured to determine a first metric value using first robotic system data, wherein the first robotic system is generated by a first robotic system used to perform a first medical procedure in a first medical environment, the prediction system comprising:
one or more processors running a correlation function, the one or more processors coupled with memory, to:
identify, via the correlation function, correlations between first features of second robotic system data and second features of efficiency-related data comprising one or more second metric values, wherein the second robotic system data and the efficiency-related data are determined for a plurality of second medical procedures in a plurality of second medical environments, wherein the second robotic system data is generated by a plurality of second robotic systems used to perform the plurality of second medical procedures in the plurality of second medical environments, and wherein the one or more second metric values is determined based at least in part on the second robotic system data and medical environment data generated by sensors deployed within the plurality of second medical environments;
predict, by the correlation function and based at least in part on the correlation and the first robotic system data, the first metric value for a time segment of the first medical procedure; and
provide the first metric value for display on a User Interface (UI).

19. The prediction system of claim 18, wherein
the first features of the second robotic system data comprises at least one of tokens, tensors, or embeddings extracted from the second robotic system data;
the second features of the efficiency-related data comprises at least one of tokens, tensors, or embeddings extracted from the efficiency-related data;
the correlations between the first features of second robotic system data and the second features of efficiency-related data is identified based at least in part on or conditioned upon metadata of the plurality of second medical procedures;
the metadata comprises at least one of identifying information of the plurality of second medical procedures, identifying information of the plurality of second medical environments in which the plurality of second medical procedures is performed, identifying information of medical staff by which the plurality of second medical procedures is performed, experience level of the medical staff, patient complexity, patient health parameters or indicators, or identifying information of the second robotic medical systems or instruments used in the plurality of second medical procedures.

20. The prediction system of claim 18, wherein
identifying, via the correlation function, the correlations between the first features of the second robotic system data and the second features of the efficiency-related data comprises identifying, via the correlation function, the correlations between the first features of the second robotic system data and the one or more second metric values for the second medical procedures;
the one or more second metric values are for each of a plurality of segments of each of the second medical procedures determined based at least in part of depth data generated by depth acquiring sensors;
the correlation function comprises one or more machine learning models;
the one or more machine learning models are trained using the second robotic system data and the efficiency-related data of the plurality of second medical procedures.
